(19) United States
(12) Patent Application Publication
(10) Pub. No.: US 2022/0390438 A1
(43) Pub. Date: Dec. 8, 2022

(54) Identification and Characterisation of Herbicides and Plant Growth Regulators

(71) Applicant: Oxford University Innovation Limited, Oxford (GB)

(72) Inventors: Clement Champion, Oxford (GB); Liam Dolan, Oxford (GB); Sarah Attrill, Oxford (GB)

(21) Appl. No.: 17/773,907

(22) PCT Filed: Nov. 4, 2020
(86) PCT No.: PCT/IB2020/060331

§ 371 Date: May 3, 2022

(30) Foreign Application Priority Data
Nov. 4, 2019 (AU) 2019904145

(51) Int. Cl. G01N 33/50; C12Q 1/6895

(52) U.S. Cl. CPC G01N 33/5097; C12Q 1/6895; C12Q 2600/13; C12Q 2600/156

(57) ABSTRACT

The present invention provides high-throughput methods capable of screening compounds for herbicidal activity or plant growth regulating activity and further allows for the prediction of the mode of action of herbicidal compounds or plant growth regulators. The methods provided herein also facilitate the identification of mutation/s responsible for resistance to herbicides in plants and the identification of the herbicide target. The methods further provide for the identification of mutation/s responsible for plant growth regulation and the identification of the plant growth regulator target.

### TECHNICAL FIELD

The present invention relates generally to the field of herbicides and plant growth regulators. More specifically, the present invention relates to methods facilitating any one of more of herbicide and plant growth regulator discovery, the prediction of herbicide or and plant growth regulator mode of action, the identification of herbicide or plant growth regulator targets, and/or the identification of mutations conferring resistance to herbicides or plant growth regulators.

### Background to the Invention

The following discussion of the background of the invention is merely provided to aid the reader in understanding the invention, and is not admitted to describe or constitute prior art to the invention.

Prior to the introduction of herbicides, many farmers relied on hand and mechanical weed control measures.

Hand weed control methods are extremely labour intensive and have detrimental social costs, while mechanical weed control measures such as tilling create environmental problems due to damage to soil structure and exposure to erosion, and often encourage further invasion by weeds. Burning may prevent the further spread of weeds if carried out before seed is set and can be undertaken over a wide area with minimal human input, but as with tilling, also exposes the soil surface to erosion.

Crop protection chemicals such as herbicides have been important tools for farmers for around 70 years, beginning with the introduction of selective herbicides. Non-selective herbicides (for example, paraquat from the 1960s and glyphosate from the 1970s) have enabled the adoption of no-till (direct drilling; zero tillage) and other reduced cultivation systems for crop production to be established. If weeds are removed by herbicides before planting, then there is no need to plough to bury weeds. No-till systems can increase yields when crops are appropriately established and have many environmental and economic benefits.

The benefits of no-till systems, which are only possible with herbicides, include a reduction in labour required, less soil erosion, water conservation, less fuel used, reduced greenhouse gas emissions and an increase in biodiversity. Combatting climate change is especially topical. Ploughing aerates the soil excessively, causing the oxidation of organic matter. Not only does this destroy good soil structure (built-up after rotational pasture, for example), but it releases large amounts of carbon dioxide. Spraying a herbicide to burn down weeds before planting in a no-till system can reduce emissions of CO₂ by more than 80%.

Reflecting their importance worldwide, herbicides accounted for more than 40% of the global market for crop and non-crop pesticides worth $64 billion in 2018. However, since the late 1990s, new herbicides have reached the market at a much slower rate. Many of the biggest selling and most widely used active ingredients, e.g. glyphosate, have been used by farmers for decades. A major herbicide with a novel mode of action has not been commercialised in the last three decades. This has caused a very serious problem for global crop protection as the new herbicides becoming available to farmers offer the same limited modes of action, which has led to resistant weeds.

Early herbicide discovery was mainly achieved by spraying chemicals onto whole plants in glasshouse conditions and observing changes in phenotype after a few days or weeks. One more recent approach is to simulate the binding of screen compounds to known or predicted herbicide targets; this approach is termed in silico screening. Another approach is to empirically test the inhibitory effect of screen compounds on the activity of known or predicted herbicide targets isolated from their biological context; this approach is called in vitro screening. Yet another approach is to empirically test the inhibitory effect of screen compounds on the growth or viability of a relevant living system; this approach is called in vivo screening. In the case of herbicide discovery, the target organisms for in vivo screening are generally whole weed plants. However, the quality of the output data produced by in vivo herbicide screening platforms where whole weed plants are exposed to screen compounds and their growth/viability is scored comes at the expense of a lower throughput than in vitro or in silico approaches.

There is an unmet need for new herbicides and in particular those with novel modes of action. Apart from offering a much needed alternative to existing herbicides with modes of action to which weeds have developed resistance, knowledge of the mode of action of a herbicide can also assist in predicting safety to humans, wildlife, and the environment in general.

### SUMMARY OF THE INVENTION

The present invention meets at least one unmet need in the art by providing methods for screening candidate compounds for herbicidal activity or plant growth regulating activity

Additionally or alternatively, the methods may be used to identify the mode of action of known or newly-identified herbicidal compounds or plant growth regulators. The methods may also be used to identify mutation/s causative of certain phenotypes in plants including, for example, herbicide resistance or plant growth modulation.

Quality of output data and the rate of throughput are two key parameters of herbicide screening platforms, and striking an effective comprise between them is a major challenge. The present inventors have developed highly effective in vivo herbicide screening methods with high throughput capabilities. The methods utilise a screening platform based on spores and/or sporelings such as, for example, those from non-vascular plants (e.g. liverworts) to screen candidate compounds for herbicide activity. Apart from the identification of new compounds with herbicidal activity or plant growth regulating activity, the methods can be utilised to predict or determine the mode of action of newly-identified and/or known herbicidal compounds or plant growth regulators. Currently, the herbicide industry uses the dicot Arabidopsis thaliana as the gold standard screening system.

Non-vascular plants such as Marchantia polymorpha have previously been considered as a possible tool for genetic screening, as detailed in Ishizaki et al., 2016 (Ishizaki, K., et al. “Molecular genetic tools and techniques for Marchantia polymorpha research”, Plant and Cell Physiology. 2016, v. 57, n. 2, pp. 262-270), as well as a genetic model, see for example Sugano et al., 2014 (Sugano, S. S., et al. ‘CRISPR/Cas9-mediated targeted mutagenesis in the liverwort Marchantia polymorpha L.’, Plant and Cell Physiology. 2014, v. 55, n. 3, pp. 475-481). However, it is not obvious to use non-vascular whole plants, spores, sporelings, explants, protoplasts or vegetative propagules in the context of screening herbicides or plant growth regulators. Whilst Marchantia polymorpha cells have been considered in a limited manner for the investigation of photosynthetic electron transport inhibitors (see Sato, F., et al. “Photoautotrophic cultured plant cells: a novel system to survey new photosynthetic electron transport inhibitors”, Zeitschrift für Naturforschung C. 1991, v. 46, n. 7-8, pp. 563-568), there are distinct challenges and fundamental biological differences that mean non-vascular whole-plants, spores, sporelings, explants, protoplasts and/or vegetative propagules would not be considered for use as a screening platform for herbicidal compounds or plant growth regulators. For example, Marchantia Polymorpha represent much physically smaller and simpler plant systems than those of unwanted weed plants, and indeed that of the current gold standard screening system Arabidopsis thaliana. In addition, non-vascular plants are structurally different in many regards to that of higher plants, for example the cuticle formed in sporelings, propagules, explants and whole non-vascular plants is chemically very different from that of higher plants. Moreover, non-vascular plants such as Marchantia polymorpha are not a logical choice for screening herbicides or plant growth regulators since Marchantia polymorpha are known to be insensitive to major herbicides such as Glycophosphate.

However, the present inventors have surprisingly found that sporelings of non-vascular plants including liverworts can be used to screen herbicides or plant growth regulators. Moreover, using non-vascular plants overcomes a number of limitations associated with the use of Arabidopsis thaliana and provides a significantly improved screening method. Given the large size and complex nature of Arabidopsis thaliana sporelings or whole plants, throughput of screening is limited by the duration of time required to grow the plants, as well as the associated additional space, resources and personnel required to cultivate the plants. For example, non-vascular plants typically only require 4 days of growth before they can be used for screening. This is compared to a typical value of 7 days for Arabidopsis thaliana. The present invention therefore provides a method with typically around at least 10 times higher throughput than an Arabidopsis-based method. In addition to the throughput limitations, using Arabidopsis thaliana sporelings or whole plants hinders fluorescence imaging based approaches to high-content screening of herbicides or plant growth regulators. Fluorescence imaging based approaches benefit from the imaged object fitting in the smallest horizontal volume of space possible and the size and complexity of Arabidopsis thaliana prohibits their use in such approaches. Non-vascular plants are advantageous for use in the methods of the present invention given their small size, simple body plan, sensitivity to herbicides with different modes of action, and/or susceptibility to genetic manipulation. This results in a higher-throughput, less expensive and more efficient screening system for candidate compounds, such as herbicides and plant growth regulators, compared to the currently existing more complex screening systems such as Arabidopsis thaliana.

In a first aspect, the present invention provides a method of screening candidate compounds for herbicidal activity or plant growth regulating activity, the method comprising the steps of:

(i) contacting a series of different candidate compounds with a plurality of test samples from non-vascular plants; and
(ii) determining whether the test samples provide a phenotypic response to said series of different candidate compounds by comparison to phenotypes of control samples from non-vascular plants not contacted with candidate compounds;

wherein the test samples and the control samples comprise whole-plants, spores, sporelings, explants, protoplasts or vegetative propagules, and the phenotypic response is indicative of the herbicidal activity or the plant growth regulating activity.

In one embodiment, the candidate compounds are candidate compounds for herbicidal activity.

In another embodiment, the non-vascular plant is a moss, hornwort or liverwort.

In a further embodiment, the test samples and control samples are sporelings.

In an additional embodiment, the test and control sporelings originate from spores of the same species of non-vascular plant.

In one embodiment, the test sporelings are moss sporelings, liverwort sporelings, hornwort sporelings, or any combination thereof.

In another embodiment, each member of the series of different candidate compounds is contacted with a different test sample.

In another embodiment, multiple members of the series of different candidate compounds are contacted with a single test sample.

In still another embodiment, the test samples and control samples are leafy liverwort sporelings, simple thalloid liverwort sporelings, complex thalloid liverwort sporelings, or any combination thereof.

In one embodiment, the test samples and control samples are selected from the group consisting of: Marchantia alpestris sporelings, Marchantia aquatica sporelings, Marchantia berteroana sporelings, Marchantia carrii sporelings, Marchantia chenopoda sporelings, Marchantia debilis sporelings, Marchantia domingenis sporelings, Marchantia emarginata sporelings, Marchantia foliacia sporelings, Marchantia grossibarba sporelings, Marchantia inflexa sporelings, Marchantia linearis sporelings, Marchantia macropora sporelings, Marchantia novoguineensis sporelings, Marchantia paleacea sporelings, Marchantia palmata sporelings, Marchantia papillate sporelings, Marchantia pappeana sporelings, Marchantia polymorpha sporelings, Marchantia rubribarba sporelings, Marchantia solomonensis sporelings, Marchantia streimannii sporelings, Marchantia subgeminata sporelings, Marchantia vitiensis sporelings, Marchantia wallisii, Marchantia nepalensis, and any combination thereof.

In one embodiment, a plurality of test sporelings are provided in a series of different wells, each well a comprising between: 400-800/mL sporelings, 300-900/mL sporelings, or 200-1000/mL sporelings.

In a further embodiment, the test samples and/or the control samples have been engineered to express a fluorescent molecule.

In one embodiment, the control samples are positive controls.

In a further embodiment, the positive controls are contacted with a known herbicide or plant growth regulator.

In one embodiment, the control samples are negative controls.

In another embodiment, the negative control samples are not contacted with a known herbicide or plant growth regulator.

In an additional embodiment, step (ii) of the method comprises comparing phenotypes of the test samples to phenotypes of positive control samples contacted with known herbicidal or plant growth regulator compounds and further comprises comparing phenotypes of the test samples to phenotypes of negative control samples not contacted with known herbicidal or plant growth regulator compounds.

In an additional embodiment, the known herbicidal compounds have a known mode of action, and said comparing of test sample phenotypes to positive control sample phenotypes is used to predict the mode of action of a candidate compound identified to have herbicidal or plant growth regulating activity.

In a further embodiment, the test samples, the negative control samples, and the positive control samples are sporelings that originate from spores of the same species of non-vascular plant.

In a further embodiment, the test samples, the negative control samples, and the positive control samples have been engineered to express a fluorescent molecule.

In one embodiment, step (ii) of the method comprises measuring the phenotypic response of the test samples after growing them in suitable media with said candidate compounds under suitable conditions for a time period of: between 1 and 3 days, between 1 and 5 days, between 3 and 6 days, between 3 and 5 days, between 2 and 3 days, between 1 and 10 days, less than 5 days, less than 4 days, or less than 3 days, after said contacting, and wherein the phenotypes of said control sporelings are determined after an equivalent time period of growth in said suitable media under said suitable conditions.

In one embodiment, step (ii) of the method comprises obtaining measurements of any one or more of: sample length, sample width, sample shape, sample pigmentation, sample circularity, sample chlorophyll concentration, and/or number of cells per sample.

In a further embodiment, the measurements are digitally recorded.

In another embodiment, comparing the phenotypic response of the test samples to any said control sample phenotypes comprises any one or more of: distributed stochastic neighbour embedding, principal component analysis (generalised weighted lease squares), principal component analysis (minimized weighted chi square), principal component analysis (minimum residuals), common factor analysis (principal axes), common factor analysis (maximum likelihood) or common factor analysis (weighted least squares).

In a further embodiment, the candidate compounds are selected as potential herbicides using an artificial intelligence algorithm, such as a Random Forest algorithm or a neural network algorithm.

In one embodiment, step (ii) of the method comprises:

obtaining phenotypic measurements from the test samples and any said control samples and thereby generating a dataset, and

using at least at least 50%, at least 60%, at least 70%, at least 80%, at least 90%, at least 95% or 99% of the dataset as a training set for the artificial intelligence algorithm.

In one embodiment, the control samples comprise positive control samples, and the artificial intelligence algorithm is used to predict a mode of action of any said candidate compounds.

In another embodiment, the method further comprises step (iii) of:

(a) contacting a candidate compound identified to have herbicidal or plant growth regulating activity in steps (i) and (ii) with a series of mutagenized samples comprising whole-plants, spores, sporelings, explants, protoplasts or vegetative propagules, wherein the test and mutagenised samples are from the same species of non-vascular plant;
(b) extracting DNA from a resistant mutagenised sample which survives said contacting in (a), or which does not exhibit growth abnormalities after said contacting in (a);
(c) sequencing the genome or a genomic portion of the resistant mutagenised sample to thereby obtain a mutagenised sample DNA sequence;
(d) aligning the mutagenized DNA sequence obtained in (c) to a reference DNA sequence and identifying a first set of sequence mismatches between the mutagenised sample DNA sequence and the reference DNA sequence;
(e) aligning a DNA sequence from a first comparison sample to said reference DNA sequence and identifying a second set of mismatches between the first comparison DNA sequence and the reference DNA sequence; and
(f) filtering the first set of mismatches with respect to the second set of mismatches to identify a first subset of mismatches that are unique to the first set of mismatches, wherein the first subset of mismatches are candidate mutations that may confer resistance to herbicides or to plant growth regulators;

wherein the first comparison sample is from an independent sample that does not survive contacting with the candidate compound or which exhibits growth abnormalities after contacting the candidate compound, and is of the same genus as the resistant mutagenised sample, and wherein the reference DNA sequence is a known reference sequence of a plant of said genus.

In another embodiment, the method further comprises: (e-i) aligning a DNA sequence of a second comparison sample to the reference DNA sequence and identifying a third set of mismatches between the second comparison sample and reference DNA sequence; and

(f) filtering the first set of mismatches with respect to the third set of mismatches to facilitate identification of a second subset of mismatches that are unique to the first set of mismatches, and generating a third subset of mismatches by filtering the first subset of mismatches with respect to the second subset of mismatches, wherein the first and second subsets of mismatches are candidate mutations that may confer resistance to herbicides or resistance to plant growth regulators;

wherein the second comparison sample is from an independent sample that does not survive contacting with the candidate compound or which exhibits growth abnormalities after contacting the candidate compound, and is of the same genus as the mutagenised samples.

In another embodiment, the mutagenised samples are M1 samples.

In another embodiment, the mutagenised samples comprise a non-naturally occurring mutation.

In a further embodiment, the method does not comprise a step of segregation analysis, complex segregation analysis or bulk segregation analysis.

In one embodiment, the aligning of (e) comprises aligning the DNA sequence of 2, 3, 4, 5, 6, 7, 8, 9, 10, 11, 12, 13, 14, 15 or more comparison samples to the reference DNA sequence and identifying the second set of sequence mismatches between the two sequences.

In another embodiment, the method further comprises filtering the candidate mutations with biological filters.

In one embodiment, the mutagenised samples are haploid.

In one embodiment, the candidate mutations are in a gene encoding a protein that is targeted by the candidate compound identified to have herbicidal or plant growth regulating activity.

In another embodiment, step (iii) is implemented using a computer.

In another embodiment, the method further comprises identifying a plant molecule or biological pathway that is targeted by a candidate compound identified to have herbicidal activity by the method, using any one or more of: enzymatic assays, chlorophyll fluorescence kinetics assays, photosynthetic oxygen evolution assays, electrolyte leakage assays, radiometric assays, spectrophotometric assays, fluorometric assays, absorbance assays, colorimetric assays, mass spectrometry, mitotic index analysis, quantitative PCR analysis, transcriptomic profiling, proteomic profiling, genome wide analysis, and/or quantitative trait locus analysis, in silico docking studies, chemical structure analysis.

In one embodiment, the plurality of test samples does not contain whole plants.

### Definitions

Certain terms are used herein which shall have the meanings set forth as follows.

As used in this application, the singular form “a”, “an” and “the” include plural references unless the context clearly dictates otherwise. For example, “a compound” as used herein also encompasses a plurality of compounds unless otherwise stated.

As used herein, the term “comprising” means “including”. Variations of the word “comprising”, such as “comprise” and “comprises” have correspondingly varied meanings. For example, a composition “comprising” material A may consist exclusively of material A, or may include material A and any other number of other additional component/s (e.g. material B, and/or material C).

As used herein, the term “plurality” means more than one. In certain specific aspects or embodiments, a plurality may mean 2, 3, 4, 5, 6, 7, 8, 9, 10, 15, 20, 25, 30, 35, 40, 45, 50, 60, 70, 80, 90, 100, 250, 300, 350, 400, 450, 500, 600, 700, 800, 900, 1000, 1250, 1500, 1750, 2000, 2500, 3000, 3500, 4500, 5000, 6000, 7000, 8000, 9000, 10000, 15000, 20000, 30000, 40000, 50000, 60000, 70000, 80000, 90000, 100000, 200000, 300000, 400000, 500000, 600000, 700000, 800000, 900000, 1000000 or more, and any numerical value derivable therein, and any range derivable therein.

As used herein, the term “between” when used in reference to a range of numerical values encompasses the numerical values at each endpoint of the range.

As used herein, “high-throughput screening” (HTS) refers to a method in which a plurality of synthetic compounds or natural products are screened for activity against one or more biological targets. HTS may be used to identify compounds with a range of biological activities, for example, drugs, pesticides, herbicides, and the like.

As used herein, the term “non-vascular plant” refers to a plant lacking a vascular system (i.e. a xylem and phloem). “Non-vascular plants” will be understood herein to encompass whole non-vascular plants, component/s of whole non-vascular plants, spores of whole non-vascular plants, and whole non-vascular plant sporelings. Non-limiting examples of non-vascular plants include bryophytes such as mosses, liverworts and hornworts.

As used herein, “whole-plant” refers to the complete plant, in particular a complete non-vascular plant. A whole-plant can also be used to refer to a miniaturized plant or a juvenile plant (i.e. a plant that has not yet reached its adult form and may therefore not be fully mature or not yet sexually mature). In the case of Marchantia polymorpha, a whole plant can refer to a gametophyte with a rooting system, one or more meristems, a photosynthetic tissue, an epidermis, a cuticle, one or more gemma cups and one or more gametangiophore (i.e. a reproductive organ). For a Marchantia polymorpha juvenile plant or miniaturized plant, the plant has a subset of the tissues, organs and features of the complete plant, including at least a rooting structure, a photosynthetic tissue and one or more meristem.

As used herein, a “weed” will be understood to refer to a plant growing in detrimental competition with cultivated plants for any one or more of water, light, nutrients and/or space. Non-limiting characteristics of weeds include low or no economic value compared to the cultivated plants, the provision of ecological and/or economic damage, vigorous growth characteristics, and/or the production of large seed numbers.

As used herein, the terms “herbicide” and “herbicidal compound” refer to a synthetic compounds or natural products capable of killing or inhibiting the growth of plants, plant cells, plant seeds or plant tissue, including but not limited to weeds and seeds thereof.

As used herein, the term “plant growth regulator” refers to a compound (natural or organic) that modulates plant growth, for example to accelerate, promote, speed up, slow down, inhibit, delay or otherwise alter plant growth, development or maturation of a plant.

As used herein, the term “phenotype” will be taken to mean a set of observable characteristics of an individual, for example, an individual plant, or a part of said plant. The term “phenotypic response” is be taken to mean the observable response of an individual, for example, an individual plant, or a part of said plant in response to a unique environment, for example in response to exposure to a candidate compound such as a candidate herbicide or candidate plant growth regulator.

As used herein, a “reference DNA sequence” refers to the reference genome sequence of a given plant. Reference DNA sequences are publicly available, for example, on databases and the like.

As used herein, a “mismatch” refers to a difference in the sequence of a read (e.g. a portion of a DNA sequence of a plant being tested to identify a causative mutation) compared to a portion of a reference DNA sequence the read best aligns to.

As used herein, the term “causative mutation” will be taken to mean a mutation causing or contributing to a phenotype of interest, for example, a mutation causing or contributing to herbicide resistance.

As used herein, “M0” denotes a plant population (i.e. the parent population) in a mutagenesis experiment prior to exposure to a mutagen. “M1” as used herein is a notation referring to the same plant population (i.e. the M0 population) following exposure to a mutagen. “M2” as used herein refers to M1 progeny following selfing (i.e. the process of crossing the mutant with itself).

As used herein, “segregation analysis” refers to a statistical technique for fitting formal genetic models to data on expressed trait or disease phenotypes in biological family members, in order to determine the most likely mode of inheritance for the trait or disease under study. Segregation analysis requires multiple generations of family members in order to determine the inheritance patterns of the phenotype being analysed.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### BRIEF DESCRIPTION OF THE FIGURES

Embodiments of the present invention will now be described, by way of example only, with reference to the accompanying Figures as set out below:

FIG. 1 is a Far-Red fluorescence micrograph showing 50 Marchantia polymorpha sporelings growing in one well of a 96-well assay plate. The sporeling density was adjusted to include a maximum number of sporelings and minimise the number of sporelings growing in contact with one another. The former increased the statistical significance of the assay while the latter was required to generate accurate data describing the response of the whole plant to the herbicide treatment.

FIG. 2 provides 10× micrographs of 5-day old Marchantia polymorpha sporelings. In the image on the left, transmitted light was recorded (Brightfield image). In the middle image, only the Far-Red photons were recorded by the camera chip (Far-Red image). In the image on the right, only the Cyan photons were recorded (Cyan image). The images were cropped to show a single sporeling; the same Marchantia polymorpha sporeling is shown in all three images. Images generated using third party software FIJI.

FIG. 3 provides linked 10× micrographs of a 5-day old sporeling. Left, the Far-Red micrograph is overlaid with the outlines of the object segmented using the Brightfield image (purple line), the Far-Red image (red line) and the Cyan image (blue line). Middle, Cyan image. Right, Brightfield image. Images generated using third party software GE Developer.

FIG. 4: provides 10× micrographs of a 1-day old Marchantia polymorpha proMpEF1a::YFP-NLS(3×) sporelings. Left, the transmitted light micrograph shows an ungerminated spore (arrow) and a 2-celled sporeling. Middle, the fluorescence micrograph shows the two nuclei of the 2-celled sporeling. The images were generated using third party software FIJI. Right, the drawing provides shows the outline of the nuclei segmented using third party software GE Developer.

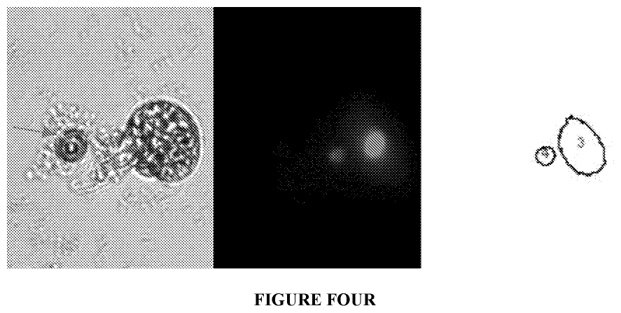

FIG. 5 provides the distribution of 10 variables from a low-resolution dataset (2×) before scaling (upper panel) and after scaling (lower panel).

FIG. 6 is a screen plot for factors created from a set of 10 variables in a low resolution dataset (2×). The horizontal lines represent the recommended number of factors to form according to either the Kaiser's criterion, the Elbow method or the Joliffe criterion. Images generated using third party software StratomineR.

FIG. 7 is a hit selection graph showing the average phenotypic distance of plants in wells containing either 0.1% DMSO (negative controls, in red), the known herbicide Isoxaben at a low concentration (positive control, in green) or a chemical of unknown activity (screen compounds, in blue). The red dotted line indicates the threshold above which a compound caused a statistically significant response in treated plants and was selected as a hit. Images generated using third party software StratomineR.

FIG. 8 is a hit selection graph showing the average phenotypic distance of plants in wells containing either 0.1% DMSO (negative controls, in red), the known herbicide Isoxaben at a low concentration (positive control, in green) or a chemical of unknown activity (screen compounds, in blue). The red dotted line indicates the threshold above which a compound caused a statistically significant response in treated plants and was selected as hit. Images generated using third party software StratomineR.

FIG. 9 provides a contour plot showing the predicted coordinates of a phenotypic response of plants treated by 0.1% DMSO (red surface) or by 3 different herbicides with different modes of action. Circles represent the 20% of the dataset used for testing the phenotypic models: if the circles are located on the surface of the same color, the corresponding model was accurate. Images generated using third party software StratomineR.

FIG. 10 provides a clustering graph showing hits that belong to the same clusters in different colours.

FIG. 11 provides photographs of Petri dishes containing 14-day old sporelings growing on 1 μM, 100 μM or 1000 μM of the known herbicide Norflurazon. Sporelings have all died on 1000 μM Norflurazon. Three replicates are shown.

FIG. 12 provides photographs of Petri dishes containing 14-day old sporelings growing on 1 μM, 100 μM or 1000 μM of the known herbicide Chlorsulfuron. Sporelings have not all died on 1000 μM Chlorsulfuron but show a marked reduction in growth. Three replicates are shown.

FIG. 13 is a dose response curve of Marchantia spores mutagenised by UV-B irradiation for different amount of time. FIG. 13 provides two replicates. 50% kill was achieved by irradiating spores with UV-B for 20 s.

FIG. 14 provides darkfield micrographs of 14-day old sporelings growing on 0.1 ppm Chlorsulfuron (left and middle) with no UV-B treatment (left) or UV-B treatment. The bigger plant is a chlorsulfuron resistant mutant; it had a phenotype similar to the UV-B treated 14-day old sporeling growing on 0.1% DMSO shown in the right panel.

FIG. 15 is a process flow diagram of a method for identification of a causative mutation causing a phenotype of interest in a tested sample, according to embodiments of the present invention.

FIG. 16: Rhizoid phenotype of two-days-old Marchantia polymorpha plants. Wild-type rhizoid phenotype (A), wavy rhizoid phenotype (B). Rhizoids are cells that grow straight in the wild type (A) and wavy in some mutants (B).

FIG. 17: Dorsal epidermis phenotype of two-months-old Marchantia polymorpha plants. Wild-type epidermis phenotype (A), stretched epidermis phenotype (B). The dorsal epidermis shows air pores (A, arrowed), which in some mutants (B) are stretched.

FIG. 18: Performance of non-allelism based mutation discovery pipeline in UV4.32. A: Effect of increasing number of non-allelic mutant backgrounds on filtering efficiency. B: Number of UV4.32 mismatches in remaining after each filtering step when using 8 non-allelic UV mutant lines.

FIG. 19: Performance of non-allelism based mutation discovery pipeline in chlorsulfuron resistant mutants. Increasing the number of allelic mutant backgrounds improves the filtering efficiency. The left-most scatter box represents the number of total mismatches in a chlorsulfuron resistant mutant line before filtering out mismatches that are also observed in the resequenced wild type genome.

FIG. 20 provides linked 4× micrographs of three 5-day old sporelings. The top panel shows a plant exposed to 0.1% DMSO, the middle panel shows a plant exposed to 10 uM of Isoxaben and the bottom panel show a plant exposed to 10 uM of a test compound selected as hit. Left to right: the FarRed micrograph is overlaid with the outlines of the object segmented using the Brightfield image (outer red line) and the outlines of the main body of the plant segmented using the FarRed image (nested red line); next, the FarRed micrograph is overlaid with the outlines of the object segmented using the Brightfield image (outer red line) and the outlines of the meristem of the plant segmented using the FarRed image (nested purple line); next, the FarRed micrograph is overlaid with the outlines of the object segmented using the Brightfield image (outer red line) and the outlines of the rhizoid of the plant segmented using the FarRed image (nested green line); next, the Cyan micrograph is overlaid with the outlines of the object segmented using the Brightfield image (outer red line) and the outlines of a cyan fluorescing mass segmented using the Cyan image (nested yellow line); finally, the FarRed micrograph is overlaid with the outlines of the object segmented using the Brightfield image (outer red line) and the outlines of the chloroplasts segmented using the FarRed image (nested blue lines).

### DETAILED DESCRIPTION OF THE INVENTION

Herbicide resistance is a major issue affecting crop and pasture production worldwide. The levels of herbicide resistant weeds are only expected to increase until herbicides exhibiting different modes of action are identified and commercialised. Methods capable of identifying new compounds with herbicidal activity or plant growth regulating activity, as well as identifying the mode of action of such compounds are highly important to combat the growing levels of herbicide resistance in weeds and other similar plants.

The present invention provides high-throughput methods capable of screening compounds for herbicidal activity or plant growth regulating activity, thus affording the potential for the discovery of new herbicides or plant growth regulators. The methods further allow prediction of the mode of action of herbicidal compounds or plant growth regulators, including compounds identified by the methods described herein and any known herbicides or plant growth regulators that may not have been characterised for mode of action. Thus, the methods provided herein may be used to identify compounds, for example, herbicidal compounds or plant growth regulators, with novel modes of action. The methods provided herein also provide for the identification of mutation/s responsible for resistance to herbicides in plants (e.g. weeds) and the identification of the herbicide target. The methods provided herein also provide for the identification of mutation/s responsible for plant growth regulation and the identification of the plant growth regulator target.

Currently, screening compounds for herbicidal activity using whole plants is relatively low throughput, as the size and complexity of whole plants does not render them amenable to high-throughput phenotypic characterisation. Described herein are high-throughput screening methods using non-vascular plants capable of concurrently screening large numbers of compounds for herbicidal or plant growth regulator activity, predicting their mode of action and identifying their target. The methods described herein may also be used for the identification of causative mutations conferring resistance to herbicidal compounds.

High-Throughput Screening

The present invention provides in vivo high-throughput methods for screening compounds for herbicidal activity or plant growth regulating activity. High-throughput screening (HTS) is a technique used to sort useful compounds quickly and efficiently from a huge number of candidates for new drugs, pesticides, herbicides, etc. and may be used to identify compounds with a range of biological activities. Without limitation, HTS as contemplated herein generally includes three elements: a suitable library of compounds for screening, an assay method, and a system for handling and/or analysing the data generated by the assay.

Compound libraries for use in the methods of the present invention may be created from combinatorial chemistry or from natural products, for example, secondary metabolites from plants, animals, and/or microorganisms. In some embodiments of the present invention, natural compounds may be processed prior to inclusion in a compound library. A non-limiting example of a suitable processing technique, which is well known to those skilled in the art, is solid phase extraction. In further embodiments of the invention, combinatorial libraries to be screened may be synthesised in the compartments in which the assay is carried out, thereby providing reference addresses for candidate compounds. A range of concentrations of any given compound/s may be tested. Various solvents may be used to solubilize solid compounds. Any suitable compounds may be screened using the methods of the present invention including, but not limited to, candidate natural, synthetic, and chemical compounds.

According to the methods of the present invention, non-vascular plants may be contacted with candidate compounds, either candidate herbicide compounds or candidate plant growth regulator compounds, and their response to those candidate compounds assessed. As noted in the definitions section the term “non-vascular plants” includes whole non-vascular plants, component/s thereof, their spores, and/or their sporelings. The plants may be non-vascular plants such as, for example, liverworts, mosses, and/or hornworts.

By way of non-limiting example, the non-vascular plants may be liverworts. The liverworts may be leafy liverworts, simple thalloid liverworts or complex thalloid liverworts. Non-limiting examples of liverworts which may be used in the screening methods described herein are as follows: Marchantia alpestris, Marchantia aquatica, Marchantia berteroana, Marchantia carrii, Marchantia chenopoda, Marchantia debilis, Marchantia domingenis, Marchantia emarginata, Marchantia foliacia, Marchantia grossibarba, Marchantia inflexa, Marchantia linearis, Marchantia macropora, Marchantia novoguineensis, Marchantia paleacea, Marchantia palmata, Marchantia papillate, Marchantia pappeana, Marchantia polymorpha, Marchantia rubribarba, Marchantia solomonensis, Marchantia streimannii, Marchantia subgeminata, Marchantia vitiensis, Marchantia wallisii and Marchantia nepalensis. Further non-limiting examples of liverworts which may be used in the screening methods described herein are plants of the Jungermanniopsida (for example, plants of the Jungermanniidae or Metzgeriidae subclasses), Marchantiopsida (for example, plants of the Marchantiidae or Sphaerocarpidae sublclasses) or Haplomitriopsida classes of liverworts.

By way of non-limiting example, the non-vascular plants may be mosses. Non-limiting examples of mosses which may be used in the screening methods described herein are as follows: Physcomitrella patens or Physcomitrella readeri moss.

By way of non-limiting example, the non-vascular plants may be hornworts. Non-limiting examples of hornworts which may be used in the screening methods described herein are as follows: hornworts of the Anthoceros, Dendroceros, Folioceros, Megaceros, Notothylas and Phaeoceros genera.

In some embodiments of the invention, the non-vascular plants may be insensitive to glyphosate and/or glufosinate.

The methods of the present invention use plant matter from non-vascular plants. The methods may comprise the steps of: (i) contacting a series of different candidate compounds with a plurality of test samples; and (ii) determining whether the test samples provide a phenotypic response to said series of different candidate compounds by comparison to phenotypes of control samples not contacted with candidate compounds. The test samples and the control samples may comprise non-vascular plant matter. In one embodiment, the test samples and the control samples comprise whole-plants, spores, sporelings, explants, protoplasts or vegetative propagules from non-vascular plants. In one embodiment, the test samples and the control samples comprise spores, sporelings, explants, protoplasts or vegetative propagules from non-vascular plants. In one embodiment, the test samples and the control samples comprise spores or sporelings from non-vascular plants. In one embodiment, the test samples and the control samples comprise spores from non-vascular plants. In a preferred embodiment, the test samples and the control samples comprise spores from a liverwort plants. Suitable liverwort spores or sporelings for use in the methods of the present invention include, for example, Marchantia spores or sporelings.

Sporelings used in the methods of the present invention may have rhizoids, vegetative photosynthetic cells and/or a nascent meristem. Plants may be used for high-throughput screening at under 1 day old, under 2 days old, under 3 days old, under 4 days old, under 5 days old, under 6 days old, under 7 days old, under 8 days old, under 9 days old, under 10 days old, under 11 days old, under 12 days old, under 13 days old or under 14 days old. Alternatively, older plants may be used.

Non-vascular plants used in the methods described herein may autofluoresce (i.e. contain endogenous fluorescent molecules). The nature of the autofluorescence may indicate that the non-vascular plants contain photosynthetic pigments, photoprotective pigments, stress-induced primary metabolites, stress-induced secondary metabolites. For example, chlorophyll is a photosynthetic pigment located in chloroplast of non-vascular plants and fluoresces in the “Far Red” spectrum. For example, NADH and NADPH accumulate in stressed non-vascular plants and fluoresces in the “Cyan” spectrum. The magnitude of the autofluorescence may indicate the degree to which the fluorescing molecules accumulate in the non-vascular plants. By extension, the nature and magnitude of the autofluorescence may be indicative of various physiological responses of the non-vascular plants contacted by the test compounds. For example, chlorophyll content may be indicative of plant growth and NAD(P)H content may be indicative of chemically induced cell stress. As another example, the localization of the chlorophyll and the localization of the NAD(P)H in the cells or plants may be indicative of light processes and metabolic processes being compromised by the test compounds contact.

Non-vascular plants used in the methods described herein may be engineered to express fluorescent or luminescent cellular markers. Expression of these fluorescent or luminescent markers may be used to create digital images for use in obtaining measurements of the phenotypic response. Use of the expression of fluorescent cellular markers to create images of biological structures has been common in the art for some time. A variety of techniques exist to express a fluorescent protein in a plant cell. Exogenous nucleic acids encoding fluorescent proteins may be introduced into plant cells using standard plant transformation methods known to those skilled in the art. One commonly used method, which may be used in some embodiments of the present invention, is Agrobacterium tumefaciens transfer-DNA (T-DNA)-induced insertion mutation. Simple and highly efficient T-DNA transformation protocols have been available to those skilled in the art for many years, including, for example, the “Floral dip” method (Clough and Bent, The Plant Journal, 1998; 16(6): 735-743). The person skilled in the art will know that T-DNA transformation protocols are available for non-vascular plants (reviewed in Genetic transformation of moss plant, Jing et al, 2013, African Journal of Biotechnology; 12(3): 227-232). T-DNA-mediated insertion is random, but as the inserted DNA fragment is flanked by 25 bp border sequences (the T-DNA), primers designed from the left border of the T-DNA can be used to isolate the genomic/T-DNA sequence junction, which can then be mapped to the genome to precisely identify the chromosomal insert location. Transposon-mediated mutagenesis is commonly used in the art, with or without T-DNA, and may be used to express fluorescent proteins in the plants used in the present invention.

Other commonly used methods for introducing exogenous nucleic acids into plant cells which may be used in the present invention include, but are not limited to, cation or polyethylene glycol treatment of protoplasts (O'Neill et al. The Plant Journal, 1993; 3(5): 729-738), calcium phosphate precipitation, electroporation, microinjection, viral infection, protoplast fusion, microparticle bombardment, agitation of cell suspensions in solution with microbeads or microparticles coated with the transforming DNA, direct DNA uptake and liposome-mediated DNA uptake. Such methods are well described in a wide range of texts commonly used by those skilled in the art, for example, Glick, Methods in Plant Molecular Biology and Biotechnology, 2018; CRC Press; Sambrook et al. Molecular Cloning: a laboratory manual, 1998; Cold Spring Harbor Laboratory. CRISPR/Cas9 genome editing technology may also be used to fluorescently tag endogenous plant proteins.

A wide variety of fluorescent proteins are available commercially (see, for example, Shaner et al., Nature Methods, 2015; 2(12): 905-909), and the person skilled in the art may select marker/s for use in the present invention based on the images required. Numerous publications are available describing the use of fluorescent proteins in the imaging of a variety of plant types, plant organs, and with a range of microscopy techniques (see, for example, Berg and Beachy, Methods in Cell Biology, 2005; 85: 153-177).

In some embodiments of the present invention, the green fluorescent protein (GFP), or a modified version of the GFP, may be used as a fluorescent marker. The original GFP isolated from the jellyfish Aequorea victoria and numerous modifications to wild-type GFP to enable its expression in plants are known in the art. GFP spectral mutants such as the cyan and yellow enhanced fluorescent proteins (ECFP and EYFP) are generally divided into seven types based on the type of chromophore (see Zacharias and Tsien, Green Fluorescent Protein: Properties, Applications, and Protocols, 2006, John Wiley and Sons; 83-120). Choice of fluorophore depends on whether more than one fluorescent marker is to be used, which requires the selection of pairs that may be spectrally separated. In some embodiments of the present invention, the red fluorescent proteins (RFP) or a modified version of the RFP may be used as a fluorescent marker. Plants may be engineered to express fluorescent proteins in particular plant structures, for example RFP and derivatives.

Non-vascular plants used in the methods described herein may be stained with fluorescent cellular dyes or probes. Fluorescent cellular dyes or probes may be applied at any time during the assay to label any particular cellular compartment, any particular cell type or any particular part of the plants. In some embodiments of the present invention, such fluorescent dyes include Calcofluor White, S4B, propidium iodide, FM1-43, FM4-64, Mitotracker dyes or Hoechst dyes. Fluorescent cellular dyes or probes may also be used as ionic content indicator, including [Ca2+] or pH indicators, or redox indicators. In some embodiment of the present invention, such fluorescent dyes or probes are OxiORANGE, HySOx, HYDROP, Hydroxyphenyl fluorescin.

The screening methods of the present invention may use any suitable arrangement of compartments (e.g. wells, tubes and the like) amenable to HTS assays.

For example, a 96-well microtitre plate may be used for both automated and non-automated forms of HTS. The assays may be set up manually or by a robotic system such as a liquid-handling robot.

Different candidate compounds are screened for herbicide activity or plant growth regulating activity separately in individual compartments. A given candidate compound may be screened in a single compartment or a plurality of compartments. Alternatively, a plurality of different candidate compounds may be screened for herbicide activity or plant growth regulating activity in a single compartment. (e.g. a natural extracts library or synthetic molecule mixtures).

In some embodiments of the present invention, a solvent may be mixed with the non-vascular plants and candidate compound/s in preparation for screening. Non-limiting examples of suitable solvents include dimethyl sulfoxide (DMSO), acetone, water, methanol and ethanol. DMSO is a carrier/universal solvent with the ability to dissolve a large number of small molecules and to carry small molecules through membranes. Without wishing to be bound by theory, DMSO or another suitable solvent, surfactants, and any other suitable additives may also enhance penetration of plant cells/tissue by test compounds and help to preserve plant cells/tissue during an assay.

Additionally or alternatively, liquid or jellified nutrient media may be mixed with the non-vascular plants and candidate compound/s in preparation for screening. Non-limiting examples of suitable nutrient media include Johnson's medium, M51C, Gamborg B5 and MS media. In some embodiments Johnson's media is utilised Johnson's medium is composed of comprising Inositol (100 mg/L), Sucrose (10 g/L), KNO₃ (6000 MgSO₄ (1000 Ca(NO₃)₂*4H₂O (4000 KCl (25 μM), H₃BO₄ (10 MnSO₄*4H₂O (1 ZnSO₄*7H₂O (1 CuSO₄*5H₂O (0.25 (NH₄)₆Mo7O₂4*4H₂O (0.25 FeSO₄*7H₂O (25 Na₂EDTA (25 NH₄H₂PO₄ (600 μM), and (NH₄)₂SO₄ (400 μM).

Persons skilled in the art will be aware that the density of the non-vascular plants per well of a given assay plate may be varied in order to maximise the statistical significance of the assay, whilst avoiding overlap of the material within each well which could affect the accuracy of measurements. One exemplary embodiment of the invention uses 50-70 spores or sporelings per well of a standard 96-well microtitre plate. Alternatively, the density may be 40-80, 30-90, or 20-100 plants, spores or sporelings per well. In some embodiments, the density is 100-225 spores or sporelings/cm², 85-260 spores or sporelings/cm², 55-285 spores or sporelings/cm². Persons skilled in the art will be aware that the density of the non-vascular plants per well of a given assay plate may be increased to saturation when the material can overlap without affecting the accuracy of other measurements. For example, the saturation density may be 11400-17100 spores or sporelings/cm², 8550-19950 spores or sporelings/cm², 5700-22800 spores or sporelings/cm2 or 285-28500 spores or sporelings/cm². Such other measurements include but are not limited to spectrophotometric measurements of spores or sporeling suspensions or fluorescence measurements of autofluorescence, fluorescence of fluorescent protein or fluorescence of fluorescent dye or probe in spores or sporelings suspensions. Exposure of the non-vascular plants to light may be varied during the assays. In some embodiments, the non-vascular plants may be grown under continuous illumination. Alternatively, exposure to light may be interrupted during the assays. The illumination may be provided at wavelength/s of between 300 nm to 900 nm (e.g. 400 nm to 700 nm). The illumination may, for example, be ultraviolet (UV) light, visible light, or infrared (IR) light).

The temperature at which the non-vascular plants are grown in the assays may, for example, be less than 15° C., less than 16° C., less than 17° C., less than 18° C., less than 19° C., less than 20° C., less than 21° C., less than 22° C., less than 23° C., less than 24° C., less than 25° C., less than 26° C., less than 27° C., less than 28° C., less than 29° C., or less than 30° C. In some embodiments the temperature is between 21° C. and 24° C.

The humidity at which the non-vascular plants are grown in the assays may, for example, be in the range of 40%-80%, 45-75%, or 50%-60%.

The duration of the assay in which the non-vascular plants are grown may, for example, be less than 1 day, less than 2 days, less than 3 days, less than 4 days, less than 5 days, less than 6 days, less than 7 days, less than 8 days, less than 9 days, less than 10 days, less than 11 days, less than 12 days, less than 13 days, less than 14 days, less than 15 days, less than 16 days, less than 17 days, less than 18 days, less than 19 days, less than 20 days, less than 21 days, less than 22 days, less than 23 days, less than 24 days, less than 25 days, less than 26 days, less than 27 days, or less than 28 days. Alternatively, older plants may be used in the assays.

In one exemplary embodiment of the invention, plant sporelings (e.g. Marchantia sporelings) are grown under continuous illumination at about 23° C. for about 5 days prior to taking measurements.

During and/or upon completion of the assays, suitable comparisons can be made between test samples in which the non-vascular plants were treated with various candidate compounds and control samples in which the non-vascular plants were not treated with the various candidates compounds. The control sample(s) may be a negative control sample in which the non-vascular plants were not mixed with a herbicide or a plant growth regulator and/or the control sample(s) may be a positive control sample in which the non-vascular plants were mixed with known herbicides or plant growth regulators (e.g. herbicides or plant growth regulators having known modes of action). These comparisons can be used to determine factors including, but not limited to, whether a given candidate compound or mixture of candidate compounds has herbicidal activity, the potency of any herbicide activity observed, the phenotypic response of the non-vascular plant in response to a given candidate compound found to exhibit herbicidal activity, and/or the predicted mode of action of a given candidate compound found to exhibit herbicidal activity. Similarly, these factors can be determined in relation to plant growth regulator. Any suitable means of making comparisons between test sample, negative control and/or positive control samples known in the art may be used. For example, the comparisons may be made via visual comparison, imaging via microscopy (e.g. fluorescent microscopy) and the like. In one embodiment, the method screens for candidates with herbicidal activity and the phenotypic response is death of the non-vascular plant following exposure to the candidate compound (i.e. the plant matter dies following candidate compound exposure). In one embodiment, death is determined as death of the plant at one week post exposure. In one embodiment, death is determined as death of the plant at two weeks post exposure. In one embodiment, death is determined as death of the plant at three weeks post exposure.

In one embodiment, the method screens for candidates with plant growth regulator activity and the phenotypic response is growth of the non-vascular plant following exposure to the candidate compound (i.e. the plant matter grows following candidate compound exposure). In one embodiment, growth is determined as growth of the plant at one week post exposure. In one embodiment, growth is determined as growth of the plant at two weeks post exposure. In one embodiment, growth is determined as growth of the plant at three weeks post exposure. The skilled person understands that are numerous ways to determine plant growth, including but not limited to, measurement of plant size (diameter), density, width, diameter or height. More complex analysis of phenotypic response can be performed using high-content screening, as detailed below.

In one embodiment, a method of screening candidate compounds for herbicidal activity or plant growth regulating activity is provided, the method comprising the steps of:

(i) contacting a series of different candidate compounds with a plurality of test samples; and

(ii) determining whether the test samples provide a phenotypic response to said series of different candidate compounds by comparison to phenotypes of control samples not contacted with candidate compounds;

wherein the test samples and the control samples comprise whole-plants, spores, sporelings, explants, protoplasts or vegetative propagules from non-vascular plants and the phenotypic response is indicative of the herbicidal activity or plant growth regulating activity. In a preferred embodiment, the non-vascular plant is a liverwort, most preferably Marchantia.

In one embodiment, a method of screening candidate compounds for herbicidal activity or plant growth regulating activity is provided, the method comprising the steps of:

(i) contacting a series of different candidate compounds with a plurality of test samples; and

(ii) determining whether the test samples provide a phenotypic response to said series of different candidate compounds by comparison to phenotypes of control samples not contacted with candidate compounds;

wherein the test samples and the control samples comprise whole-plants, spores, sporelings, explants, protoplasts or vegetative propagules from liverwort plants and the phenotypic response is indicative of the herbicidal activity or plant growth regulating activity.

In one embodiment, a method of screening candidate compounds for herbicidal activity or plant growth regulating activity is provided, the method comprising the steps of:

(i) contacting a series of different candidate compounds with a plurality of test samples; and

(ii) determining whether the test samples provide a phenotypic response to said series of different candidate compounds by comparison to phenotypes of control samples not contacted with candidate compounds;

wherein the test samples and the control samples comprise whole-plants, spores, sporelings, explants, protoplasts or vegetative propagules from Marchantia and the phenotypic response is indicative of the herbicidal activity or plant growth regulating activity.

Non-limiting methods are discussed below and also presented in the Examples of the present application.

High-Content Screening

In some embodiments, the methods of the invention employ high-content screening (HCS). In general, HCS uses fluorescence or light emission measurements of samples in a high-throughput format and quantitatively analyses various parameters.

Non-limiting examples of parameters that may be used in HCS according to the methods described herein include non-vascular plant length, width, shape, pigmentation, circularity, chlorophyll content, and number of cells per non-vascular plant (note that as set out above, a “non-vascular plant” as used herein encompasses whole non-vascular plants, component/s thereof, their spores and their sporelings). Any one or more of these parameters, optionally including other parameters, comprises the phenotypic response of the plant to the compound.

Imaging may be performed by a variety of techniques. Those skilled in the art would be aware that HCS is often carried out using fully automated fluorescence imaging systems. In some embodiments of the present invention, a liquid-handling robot is incorporated into a fully automated fluorescence imaging system. In other embodiments, the assays may be set up manually prior to imaging with a fully automated fluorescence imaging system for high-throughput imaging. The fully automated fluorescence imaging system may comprise a high-throughput fluorescence microscope. Some embodiments of the present invention do not require the use of confocal microscopy.

Several images may be generated for each non-vascular plant samples utilised in a given assay. In some embodiments of the present invention, images may be generated by recording transmitted light. Additionally or alternatively, images may be generated by recording transmitted light. Images may be created using only Far Red photons and/or only Cyan photons. A 2× objective lens may be used to produce a Far-Red image or a Yellow image with a field of view covering one entire well of a 96-well microtitre plate. This image may be a Far-Red fluorescence micrograph. This image may be a Yellow fluorescence micrograph. A 4× objective lens may produce a Far-Red image, a Cyan image, a Yellow image or a Brightfield image with a field of view fitting exactly within the border of one well of a 96-well microtitre plate. A 10× objective lens may be used to produce a set of 1-9 Far-Red images, 1-9 Cyan images, 1-9 Yellow images and 1-9 Brightfield images that cover a small portion of one well, for example, 1/32^{nd}-⅓^{rd} of the well bottom surface. In some embodiments of the invention, images are overlaid with the outline of an object created by another image. Images may be overlaid with outlines created by images using the same or different photons. Images may have a field of view larger, equal to or smaller than the diameter of a well by using objective lenses with 2×, 4×, 10×, 20× or 40× optical magnification. In some embodiments of the invention, image analysis protocols are used which distinguish plants and subcellular objects such as nuclei from the background.

In some embodiments of the present invention, several images are generated at different points through the structure of the non-vascular plant. These images are referred to as slices. The slices may be under 5 μm, under 10 μm, under 20 μm, under 30 μm, under 40 μm, under 50 μm, under 60 μm, 70 μm, under 80 μm, under 90 μm or 100 μm deep. The skilled artisan can determine the number of slices required to image through a complete structure based on the sample thickness. Manual imaging may also be used to enable the person skilled in the art to formulate an automated protocol. Spatially and/or temporally related images may be combined to form a “stack” for the purposes of display and/or analysis. For example, in some embodiments of the invention, a Far Red image may be created which is a maximum intensity projection of a stack made up of 5 slices, each slice being 20 μm deep. A stack may be created with 2, 3, 4, 5, 6, 7, 8, 9, 10 or more images. A stack may also be created with under 20, under 30, under 40, under 50, under 60, under 70, under 80, under 90, under 100 images. Images may be saved in digital format. Any or all images created may be used in subsequent analysis. Some embodiments of the invention use computer scripts to add metadata to images. Metadata may include barcodes of assay plates, dates, image acquisition protocols and/or image analysis protocols. Computer scripts may record whether an image is linked to other image/s.

Phenotypic “fingerprinting” of the response of a living system to a chemical is widely used in the field of drug discovery (see, for example, Reisen et al., Assay and Drug Development Technologies, 2015; 13(7): 415-427) and software tools to assist the person skilled in the art are widely available (see, for example, Omta et al., Assay and Drug Development Technologies, 2016; 14(8): 439-452).

A phenotypic fingerprint may be consistent with the known effect of a compound. For example, a compound that inhibits pigment synthesis may cause the contacted plants to produce fewer pigments and grow smaller; the fingerprint may thus be a quantitative representation of this anticipated plant response as described by several phenotypic variables. A phenotypic fingerprint may also be unexpected. For example, a compound that inhibit photosynthesis may lead to cellular elongation and a shift in the cellular localization of the chloroplasts; the fingerprint may thus also be a quantitative representation of a surprising plant response as described by several phenotypic variables.

In some embodiments of the invention, data is normalised by the median of the negative controls at the plate level to minimise noise across plates. In further embodiments, distribution of variables is checked for normality and transformed, if necessary. Transformation of the data may be recommended by the software used for analysis, which may also recommend a data transformation method and/or automatically execute the transformation. Non-limiting examples of data transformation methods which may be suitable for use with the present invention include: square root, power of 2, power of 3, log, log 2, log 10, inverse. Variables may then be scaled to equalize the weight of variables with different means in downstream analysis steps. In some embodiments of the invention, scaling is carried out using the Z-score method at the plate level. Screen level scaling may also be possible at this step.

No particular limitation exists in relation to statistical methods used to analyse the data. Many software packages are suitable for use with the methods of the present invention which provide a data analysis pipeline where each step can be customized by the person skilled in the art by changing the statistical methods and parameters used. In some embodiments of the invention, correlated variables in the negative control and screen compound data are reduced to factors by Common Factor Analysis (e.g. distributed stochastic neighbour embedding, principal component analysis (generalised weighted lease squares), principal component analysis (minimized weighted chi square), principal component analysis (minimum residuals), common factor analysis (principal axes), common factor analysis (maximum likelihood), or common factor analysis (weighted least squares)). In further embodiments, oblique (non-orthogonal) rotation may be employed to reduce the number of factors. The number of factors retained may be automatically determined according to the Kaiser's Elbow or Joliffe's criterion. Additionally or alternatively, factors to be retained may be manually selected upon visual inspection of a screen plot

Alternatively, all or a subset of raw variables may be selected which describe a phenotypic difference between the plants treated with a candidate herbicide and a negative or a positive control.

Factors retained may be used in some embodiments of the invention to select compounds as “hits” according to the phenotypic Euclidean distance to the median of the negative control in the multifactorial space. Alternatively, all or a subset of raw variables may be selected which describe a phenotypic difference between the plants treated with a candidate herbicide and a negative or a positive control. In another embodiment of the invention, a combination of raw variables and factors are retained for hit selection.

The person skilled in the art may select a level of significance above which compounds will be regarded as “hits”. In some embodiments, clustering of “hits” with positive controls may be used to predict a mode of action of a potential herbicide. In some embodiments, clustering of “hits” with positive controls may be used to predict a mode of action of a potential plant growth regulator. Clusters of “hits” and positive controls may be generated using the Ward agglomerative method where K-means are automatically selected and the distance between cluster centers of gravity is calculated as the Euclidean distance in the multifactorial space. Alternative agglomerative methods include McQuitty/Weighted Pair Group Method with Arithmetic Mean, Single agglomeration, Complete agglomeration, Centroid agglomeration or, Median agglomeration. Alternative distance calculations include Maximum distance, Manhattan distance, Canberra distance, Minkowski distance or Cosine distance. In some embodiments, if a “hit” falls outside of clusters associated with a known mode of action positive control, the hit may be visually inspected for the presence of atypical symptoms, and if atypical symptoms are observed the hit may be predicted to have a novel mode of action. In some embodiments of the invention, a “hit” cannot be predicted to be either a known or novel mode of action. In this scenario, the hit may be manually progressed to a dose-response experiment where plants are treated with a range of concentrations of the “hit” ranging from 1 nM to 50 000 nM and all resulting datapoints are processed and re-analysed according to the methods described herein. Exemplary concentrations for such a dose-response experiment may range from 1 nM to 50 000 nM.

Artificial intelligence may be used for “hit” selection. In some embodiments of the invention, at least 20%, at least 30%, at least 40%, at least 50%, at least 60%, at least 70% or at least 80% of data obtained by measuring the phenotypic response of the plant to a negative control compound are used as a training set for an artificial intelligence algorithm. The algorithm may be a Random Forest algorithm. In some embodiments of the present invention, an artificial intelligence algorithm, for example, a Random Forest algorithm, may be used for prediction of mode of action. Alternatively, a Neural Network algorithm may be used for prediction of mode of action. At least 20%, at least 30%, at least 40%, at least 50%, at least 60%, at least 70% or at least 80% of data obtained by measuring the phenotypic response of the plant to a positive control compound may be used as training data for an artificial intelligence algorithm used to predict mode of action. Statistical tests may be used to determine the probability that a “hit” matches a phenotypic model generated by artificial intelligence. In some embodiments of the invention, this decision-making step may be automated. Additionally or alternatively, this decision-making step may be carried out manually.

The present invention encompasses measuring morphological and/or physiological characteristics of non-vascular plants (e.g. test samples exposed to candidate compounds, negative and/or positive controls) to create phenotypes. The phenotypic response can be used to predict a mode of action. In some embodiments of the present invention, measurements may be recorded of under 10, under 20, under 30, under 40, under 50, under 60, under 70, under 80, under 90, under 100, under 250, under 500 or under 1000 morphological and/or physiological characteristics per individual plant. Non-limiting examples of morphological and/or physiological characteristics which may be measured include plant length, plant width, plant shape, plant pigmentation, plant circularity, chlorophyll concentration, and number of cells per plant.

In further embodiments of the invention, measurements may be recorded of under 10, under 20, under 30, under 40, under 50, under 60, under 70, under 80, under 90, under 100, under 250, under 500 or under 1000 morphological and/or physiological characteristics of control samples. The morphological and/or physiological characteristics which may be measured for the control samples also include non-vascular plant length, width, shape, pigmentation, circularity, chlorophyll concentration, and number of cells. The control sample may be a sample of the same non-vascular plant subjected to identical assay conditions as the test sample, but without the addition of the test compound. In some embodiments of the invention, DMSO may be added to the control sample in place of the test compound. In other embodiments, another solvent may be added to the control sample. The solvent added may be the solvent added to the assay wells which include the test compound.

Compounds selected as potentially having herbicidal activity or plant growth regulating activity using the methods of the invention may be referred to herein as “hits”. The difference between the phenotype of a non-vascular plant following the assay and the phenotype of the negative control non-vascular plant is referred to herein as the phenotypic response to the compound. Compounds may be selected as “hits” based on the magnitude of the phenotypic response of the non-vascular plant matter or sporeling to the compound. Additionally or alternatively, compounds may be selected as “hits” based on the nature of the phenotypic response of the non-vascular plant matter or sporeling to the compound.

In some embodiments of the invention, mode of action of “hits” may be predicted. This may be achieved by creating a phenotype for the non-vascular plant when assayed with a compound with known herbicidal activity or plant growth regulator activity. The phenotype of the plant or sporeling assayed with the test compound may then be compared to the phenotype of the non-vascular plant assayed with a compound with known herbicidal activity, referred to herein as the “positive control”. Compounds with known herbicidal activity which may be used in the methods of the present invention as a positive control include commercial herbicides used at concentrations known to cause mode of action specific symptoms. Examples of such compounds include clodinafop-propargyl, cyhalofop-butyl, diclofop-methyl, fenoxaprop-P-ethyl, fluazifop-P-butyl, haloxyfop-R-methyl, propaquizafop, quizalofop-P-ethyl, alloxydim, butroxydim, clethodim, cycloxydim, profoxydim, sethoxydim, tepraloxydin, tralkoxydim, pinoxaden, amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-Na, foramsulfuron, halosulfuron-methyl, imazosulfuron, iodosulfuron, mesosulfuron, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, imazapic, imazamethabenz-methyl, imazamox, imazapyr, imazaquin, imazethapyr, cloransulam-methyl, diclosulam, florasulam, flumetsulam, metosulam, penoxsulam, bispyribac-Na, pyribenzoxim, pyriftalid, pyrithiobac-Na, pyriminobac-methyl, flucarbazone-Na, propoxycarbazone-Na, benfluralin, butralin, dinitramine, ethalfluralin, oryzalin, pendimethalin, trifluralin, amiprophos-methyl, butamiphos, dithiopyr, thiazopyr, propyzamide=pronamide, tebutam, chlorthal-dimethyl, clomeprop, 2,4-D, 2,4-DB, 2,4-DP, MCPA, MCPB, mecoprop, chloramben, dicamba, TBA, clopyralid, fluroxypyr, picloram, triclopyr, carboxylic acid quinclorac, quinmerac, benazolin-ethyl, ametrine, atrazine, cyanazine, desmetryne, dimethametryne, prometon, prometryne, propazine, simazine, simetryne, terbumeton, terbuthylazine, terbutryne, trietazine, hexazinone, metamitron, metribuzin, amicarbazone, bromacil, lenacil, terbacil, chloridazon, desmedipham, phenmedipham, bromofenoxim, bromoxynil, ioxynil, bentazon, pyridate, pyridafol, chlorobromuron, chlorotoluron, chloroxuron, dimefuron, diuron, ethidimuron, fenuron, fluometuron, isoproturon, isouron, linuron, methabenzthiazuron, metobromuron, metoxuron, monolinuron, neburon, siduron, tebuthiuron, propanil, pentanochlor, butylate, cycloate, dimepiperate, EPTC, esprocarb, molinate, orbencarb, pebulate, prosulfocarb, benthiocarb, tiocarbazil, triallate, vernolate, bensulide, benfuresate, ethofumesate, glyphosate, sulfosate, glufosinate-ammonium, bilanaphos, amitrole, norflurazon, diflufenican, picolinafen, beflubutamid, fluridone, flurochloridone, flurtamone, clomazone, acifluorfen-Na, bifenox, chlomethoxyfen, fluoroglycofen-ethyl, fomesafen, halosafen, lactofen, oxyfluorfen, fluazolate, pyraflufen-ethyl, cinidon-ethyl, flumioxazin, flumiclorac-pentyl, fluthiacet-methyl, thidiazimin, oxadiargyl, azafenidin, carfentrazone-ethyl, sulfentrazone, pentoxazone, benzfendizone, butafenacil, pyraclonil, profluazol, flufenpyr-ethyl, acetochlor, alachlor, butachlor, dimethachlor, dimethenamid, metazachlor, metolachlor, pethoxamid, pretilachlor, propachlor, propisochlor, thenylchlor, diphenamid, napropamide, naproanilide, flufenacet, Mefenacet, fentrazamide, anilofos, cafenstrole, piperophos, DSMA, MSMA, asulam, naptalam, diflufenzopyr-Na, Dichlobenil, chlorthiamide, Isoxaben, flupoxam, diquat, paraquat, chlorpropham, propham, carbetamide, Dinitrophenol DNOC, dinoseb, dinoterb, Flamprop-M-methyl /-isopropyl, quinclorac, TCA, dalapon, flupropanate, difenzoquat, mesotrione, sulcotrione, isoxachlortole, isoxaflutole, benzofenap, pyrazolynate, pyrazoxyfen, Benzobicyclon, bromobutide, (chloro)-flurenol, cinmethylin, cumyluron, dazomet, daimuron, etobenzanid, fosamine, indanofan, metam, oxaziclomefone, oleic acid, pelargonic acid, pyributicarb. The person skilled in the art may select any herbicide with a known mode of action for use as a positive control in the methods. Similarly, compounds with known plant growth regulation activity which may be used in the methods of the present invention as a positive control include commercial plant growth regulators used at concentrations known to cause mode of action specific symptoms. The person skilled in the art may select any plant growth regulator with a known mode of action for use as a positive control in the methods.

Target Identification

The methods of the present invention may include analyses for the identification of targets (e.g. protein target/s) of herbicides such as, for example, the targets of candidate compounds screened and identified to have herbicidal activity and/or of known herbicides where the mode of action is unknown.

In some embodiments, the target identification may involve contacting “hits” identified by the methods of the invention with non-vascular plants that have been mutagenised. DNA may then be extracted from the plants that survive said contacting. Mutagenesis methods are standard in the art. The mutagen may, for example, be radiation. In some embodiments, the mutagen is selected from the group consisting of ultra-violet (UV) light, x-ray, gamma rays and neutrons. In further embodiments, the mutagen may be UV light, which could be UV-A, UV-B or UV-C light. Additionally or alternatively, mutagenesis may be carried out using chemical agents. Non-limiting examples include alkylating agents such as ethyl methanesulfonate (EMS). In some embodiments, dimethyl sulphate, sodium azide, or methylnitronitrosoguanidine (MNNG) may be used to introduce mutations into a non-vascular plant. The chemical agent may also be a deaminating agent or an intercalating agent. In further embodiments of the invention, the mutagen is a transposable element.

DNA extraction methods are standard in the art. DNA may be extracted using phenol, chlorophorm and isoamyl alcohol. Other well-known DNA extraction techniques include enzymatic methods, silica-(spin) column based methods, anionic resins, methods using magnetic beads and the CaCl density gradient DNA extraction method. Cetyl trimethylammonium bromide (CTAB) and 2-β-mercaptoethanol are commonly used for plant DNA extraction where plant tissue contains high levels of polysaccharides, polyphenols and/or other secondary metabolites (see, for example, Clark, Plant molecular biology—a laboratory manual, 1997; Springer: 305-328). DNA may be extracted from the whole mutant non-vascular plant or a subpart of the whole mutant non-vascular plant, or a mutant spore, sporeling, explant, protoplast or vegetative propagule of the mutant non-vascular plant.

In some embodiments of the invention, a genomic DNA library is prepared. In further embodiments, the library is then sequenced. Any high-throughput sequencing technology that is capable of sequencing the entire genome of a plant may be used, including technologies based on clonal amplification, technologies based semiconductors and single-molecule real-time (SMRT) sequencing (for a recent review of potentially suitable commercially available platforms, see Reuter et al., Molecular Cell, 2015; 58: 586-597). Raw sequencing reads may then be “trimmed” to remove poor quality sequence and/or artefacts of the sequencing process such as primers and sequencing adaptors. Any suitable known software program, such as, for example, Trimmomatic may be used. Trimmomatic trims Illumina sequencing adapters and parts of reads associated with poor sequencing quality. Other known processes for carrying out quality trimming may also be used.

In some embodiments of the invention, the read files may be interleaved. Interleaving may be carried out using any suitable parsing script. For example, where paired reads are obtained by the sequencing system, the parsing script may be used to reunite the two mate pairs of all paired reads into a single file.

Some embodiments may include a normalisation step. The normalisation process may, for example, be carried out by normalising by 31-mers using a script that calls any suitable known software program, such as, for example, Khmer. In this example, the normalisation program looks at the distribution of k-mers in all reads using a predefined value fork and discards a proportionate amount of reads containing the most frequent k-mers for the reason that they only provide redundant information. This step may be performed to make the alignment process more memory efficient.

The normalised reads file may then be de-interleaved or decoupled using any suitable parsing script that separates the two mate pairs of all paired reads in two files. This step is the opposite of the interleaving step. For each paired read, there are two mates identified as belonging to the same paired read. They can either be written in the same file (i.e. interleaved), or in separate files (deinterleaved). The process of going from one to another is merely parsing according to a tagging string that identifies mates as belonging to the same paired read. This tagging originates from the files produced by the sequencing platform and may look, for example, like XYZ/1 for mate 1 and XYZ/2 for mate 2. The software identifies them by text matching and writes the corresponding DNA sequence to either the same files or two separate files.

The sequenced genome may then be aligned to a reference genome for the plant. The reference DNA sequence may be a known reference sequence for a plant of the genus. Reference DNA sequences are published on publicly available databases. The entire genome sequence is publicly available for many non-vascular plants including, for example, liverworts such as Marchantia (reference sequences for the nuclear genome and organellar genomes are publicly available for Marchantia).

The reference DNA sequence may be aligned to a further comparison sequence in some embodiments of the invention. The comparison sequence may be from an independent plant of the same genus that does not survive contacting with the compound. In some embodiments, the methods of the present invention may involve obtaining a set of mismatches between the DNA sequence of the mutant plant and the reference DNA sequence. A second set of mismatches may then be obtained between the reference DNA sequence and the comparison sequence. Further embodiments of the invention may then involve filtering the first set of mismatches with respect to the second set of mismatches to identify a subset of mismatches that are unique to the first set of mismatches. The subset of mismatches may be candidate mutations for a causative mutation for herbicide resistance or plant growth modulation. This may help to identify targets of novel herbicides or plant growth regulators identified by the methods of the invention.

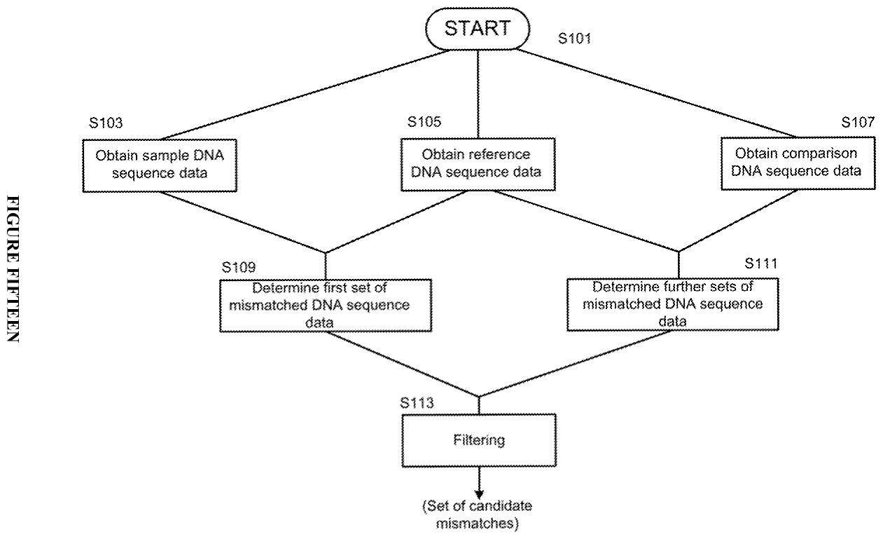

The method for identification of a causative mutation causing a phenotype of interest in a tested sample, according to embodiments of the present invention, is provided in FIG. 15.

In one embodiment, target identification to identify a mutation associated with a phenotype of interest in a non-vascular plant is performed according to the following method:

(a) aligning the DNA sequence of a test sample to a reference DNA sequence and identifying a first set of sequence mismatches between the two sequences;

(b) aligning the DNA sequence of at least one comparison sample to the reference DNA sequence and identifying a second set of sequence mismatches between the two sequences;

(c) filtering the first set of mismatches with respect to the second set of mismatches to identify a subset of mismatches that are common to the first and second sets of mismatches, wherein the subset of mismatches are candidate mutations for the causative mutation;

wherein the test sample and the comparison sample(s) are from independent non-vascular plants exhibiting the phenotype of interest and wherein the independent non-vascular plants are the same genus; and

wherein the reference DNA sequence is a known reference sequence for a non-vascular plant of the genus.

In one embodiment, target identification to identify a mutation associated with a phenotype of interest in a non-vascular plant is performed according to the following method:

(a) aligning the DNA sequence of a test sample to a reference DNA sequence and identifying a first set of sequence mismatches between the two sequences;

(b) aligning the DNA sequence of at least one comparison sample to the reference DNA sequence and identifying a second set of sequence mismatches between the two sequences;

(c) filtering the first set of mismatches with respect to the second set of mismatches to identify a subset of mismatches that are unique to the first set of mismatches, wherein the subset of mismatches are candidate mutations for the causative mutation;

wherein the test sample is from a non-vascular plant exhibiting the phenotype of interest and wherein the comparison sample is from an independent non-vascular plant of the same genus that does not exhibit the phenotype of interest; and

wherein the reference DNA sequence is a known reference sequence for a non-vascular plant of the genus.

In one embodiment the test sample is biological matter and/or at least one comparison sample from a non-vascular land plant, wherein the non-vascular plant is a bryophyte. In one embodiment, the test sample and/or at least one comparison sample is biological matter from a bryophyte selected from the group consisting of moss, liverwort and hornwort. In one embodiment, the test sample and/or at least one comparison sample is biological matter leafy liverwort, simple thalloid liverwort or a complex thalloid liverwort. In one embodiment, the test sample and/or at least one comparison sample is biological matter from a plant of the Marchantia species.

In some embodiments of the invention, the DNA sequence of an additional comparison sample may be aligned to the reference DNA sequence, thereby identifying a third set of sequence mismatches between the two sequences. The first set of mismatches may then be filtered with respect to the third set of mismatches to identify a subset of mismatches that are common to the first and third sets of mismatches. The two subsets of mismatches may then be candidate mutations for a causative mutation for herbicide resistance or plant growth modulation. The additional comparison sample may be from an independent plant of the same genus that does not survive contacting with the compound. Some embodiments of the invention involve aligning the DNA sequence of 2, 3, 4, 5, 6, 7, 8, 9, 10, 11, 12, 13, 14, 15 or more comparison samples to the reference DNA sequence to identify set/s of mismatches between the sequences, which may then be used to compare to the set of mismatches between the DNA sequence of the mutant plant and the reference DNA sequence to identify unique mismatches for candidate mutations for herbicide resistance or plant growth modulation. Many software packages are available to assist the skilled artisan in aligning the DNA sequences and filtering sets of mismatches.

In some embodiments of the invention, regions of the genome where more reads than expected align may be excluded. That is, a sequencing depth is defined by the number of sequencing reads from the sample that align against a region of the reference DNA sequence. When sequencing the DNA sequence of a sample, the user may select how many times to sequence the same portion of the DNA sequence. This selection defines the expected sequencing depth. For example, aiming for a sequencing depth of 1 would require the sampling system to sequence the entire DNA sequence of the sample a single time. For an expected sequencing depth of 20, the sampling system would sequence 20 times as much of the sample's DNA.

Therefore, as an example, if the observed sequencing depth at a defined position is 10, then 10 sequencing reads are aligned to a region of the reference DNA sequence that include this position. If the expected sequence depth was 1, this would suggest that 9 of the 10 reads have been aligned against this region of the DNA sequence by mistake. For this reason, the software regards any mismatch in a region of the reference DNA sequence where the observed sequencing depth is higher than the expected sequencing depth as being the likely consequence of having wrongly aligned reads and thus removes it from the set of mismatched data. In other words, the mismatch is regarded as an alignment artefact and not as a candidate mutation and so is discarded or removed from the data set. Put another way, to determine the first or further sets of mismatched DNA sequence data, the described method and software may reject at least one region of the sample DNA sequence that aligns with the reference DNA sequence based on the actual read depth being over an expected read depth. Many suitable software programs may be used to implement this functionality.

Moreover, the frequency of a mismatch occurrence in the group of reads that align at a position in the genome may be used to filter out alignment artefacts. For example, if the mutant is a diploid species, the expected frequency of the mismatch in the mutant genome is 50%, while in a haploid species it is 100%. If the observed mismatch frequency does not match the expected mismatch frequency for the defined species, the associated reads are discarded from the set of data. Again, this applies to the sets of data for both the sample and the comparison DNA sequences.

In some embodiments of the invention, the non-vascular plant that has been mutagenised is an M1 mutant. “M1” refers to the first generation of mutants, meaning that the M1 mutant is not the progeny of a mutagenised non-vascular plant. The mutagenised non-vascular plant may comprise non-naturally occurring mutations. Some embodiments of the invention do not comprise steps of segregation analysis, complex segregation analysis or bulk segregation analysis.

Additionally or alternatively, target identification may be achieved by any one or more of: enzymatic assays, chlorophyll fluorescence kinetics assays, photosynthetic oxygen evolution assays, electrolyte leakage assays, radiometric assays, spectrophotometric assays, fluorometric assays, absorbance assays, colorimetric assays, mass spectrometry, mitotic index analysis, quantitative PCR analysis, transcriptomic profiling, proteomic profiling, genome wide analysis, quantitative trait locus analysis, in silico docking studies, chemical structure analysis (some reviewed in Dayan 2015 “Biochemical markers and enzyme assays for herbicide mode of action and resistance studies”). The person skilled in the art would be familiar with all of the aforementioned techniques for downstream analysis.

### INCORPORATION BY CROSS-REFERENCE

This application claims priority from Australian provisional patent application number 2019904145 filed on 4 Nov. 2019, the entire content of which is incorporated herein by cross-reference.

### EXAMPLES

The present invention will now be described with reference to the following specific examples, which should not be construed as in any way limiting.

### Example One: Marchantia polymorpha Sporelings as a Screening System for Herbicide Activity

Preparation of Assay Plates

Front-end assays were either set up manually or by a liquid handling robot. Manual assay plate preparation started by adding 1 μL of 10% DMSO solution to the bottom of wells in 96-well microtiter plates followed by the addition of 99 μL of cell suspension in liquid nutritive media. Automated assay plate preparation started by adding 7 μL of 1.4% DMSO solution to the bottom of wells in 96-well microtiter plates followed by the addition of 93 μL of cell suspension in liquid nutritive media. Typically, one screen compound only was tested in one well and one well only was used to test a screen compound.

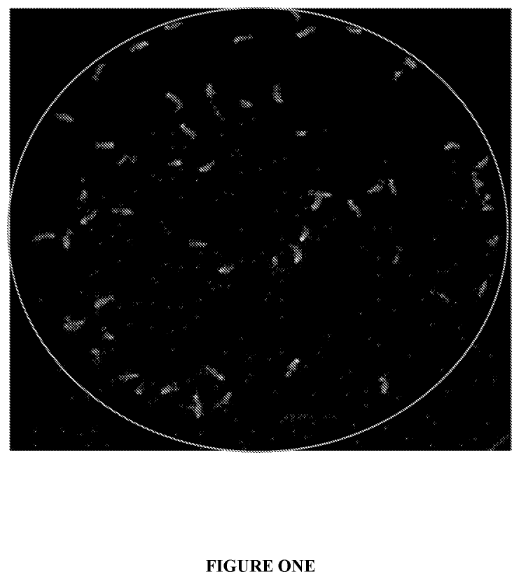

To maximize the statistical significance of the assays while avoiding overlap of sporelings, 50-70 Marchantia polymorpha spores were placed in each well of 96-well plates (FIG. 1).

Growth Conditions

Assay plates were placed in cabinets where light and temperature were controlled. Plants were grown under continuous illumination for 5 days. The temperature was set at 23 degrees Celsius. Plants were grown in Johnson's medium.

Imaging

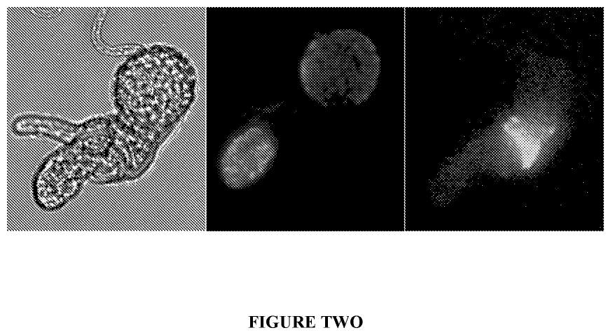

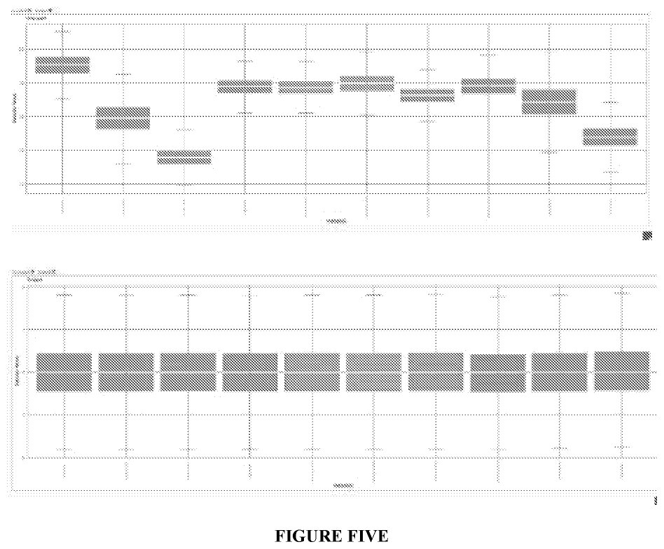

Microtiter plates containing 1-day to 5-day old Marchantia polyrnorpha sporelings grown in the presence of either a negative control (DMSO), positive controls (commercial herbicides) or screen compounds were placed in a high-throughput fluorescence microscope, the InCell Analyzer 2500 (GE Healthcare). Several images were generated (FIG. 2, FIG. 5): an image where only the Far-Red photons were recorded by the camera chip (Far-Red image), an image where only the Cyan photons were recorded (Cyan image) an image where only the Yellow photons (Yellow image) were recorded and an image where transmitted light was recorded (Brightfield image). A 2× objective lens was used to produce a Far-Red image with a field of view covering the entire well. Here, the Far-Red image was a 2D fluorescence micrograph. A 4× objective lens was used to produce a Far-Red image with a field of view fitting exactly within the border of a well. Again, the Far-Red image was a 2D fluorescence micrograph. A 10× objective lens was used to produce a set of 4 Far-Red images, 4 Cyan images, 4 yellow images and 4 Brightfield images that covered a small portion of the well (⅛th). Here, the Far-Red images were a maximum intensity projection of a 5-slices stack where each slice was 20 μm deep. The Cyan and yellow images were a maximum intensity projection of a 4-slices stack where each slice was 20 μm deep. The Brightfield images were a single 20 μm deep slice. Any or all the four channel images could be used in subsequent image analysis. Images may have a field of view larger, equal or smaller than the diameter of a well by using objective lenses with 2×, 4×, 10×, 20× or 40× optical magnification.

### Discussion

Marchantia spores, or spores of any sporulating plants, have to date not been used as a model organism for herbicide discovery. The choice of Marchantia polymorpha as a model organism for herbicide discovery was not an obvious one because Marchantia polymorpha, like liverworts in general, is known to be insensitive to the major herbicides glyphosate and glufosinate.

In order to grow large number of plants amenable to high content phenotypic characterisation the developmental stage of the Marchantia sporelings had to be finely tuned.

The Effect of Developmental Stage

Marchantia spores were grown for five days until they reached a developmental stage when a diversity of cell types and tissues representative of a whole plant had formed. Namely, 5-day old sporelings have rhizoids, vegetative photosynthetic cells and a nascent meristem. Furthermore, 5-day old sporelings presented a size and shape more convenient for microscopy than later developmental stages.

### Example Two: Identification of Herbicide Hits and Prediction of Mode of Action Using MoA Galaxy

### Background

Plants respond in a variety of ways to herbicide treatments, from hardly detectable physiological changes, minor lesions to plant death. The magnitude of the response correlates with the potency of the chemicals applied to the plant. The nature of the response differs for chemicals that act through different modes of action.

The quantification of the magnitude of the phenotypic response of plants treated with screen compounds assists the selection of hits based on herbicidal potency. Independently from the strength of the response, the nature of the symptoms exhibited by plants treated by screen compounds may be used to select hits. Taken together, the magnitude and the nature of the plants response to a screen compound are not only indicative of herbicide potency but may also be indicative of herbicide mode of action. Phenotypic fingerprinting of the response of a living system to a chemical is used in the field of drug discovery (Linking Phenotypes and Modes of Action Through High-Content Screen Fingerprints) and software tools to assist experimentalists are widely available. This approach to chemical screening is called High-Content Screening and relies on the simultaneous recording of a multitude of phenotypic descriptors, such as length, area, color, shape, etc.

Translating this approach to herbicide discovery however represents a challenge because in vivo models currently in use are hindered by their morphological complexity: 3D seedlings are more complicated objects to image than 2D cell cultures, particularly with a throughput sufficient for screening large chemical libraries. The present inventors have developed a high-content screening platform that utilises Marchantia spores' amenability to grow in close to 2D (and to be transformed with fluorescent cellular markers).

Methods

Image Analysis

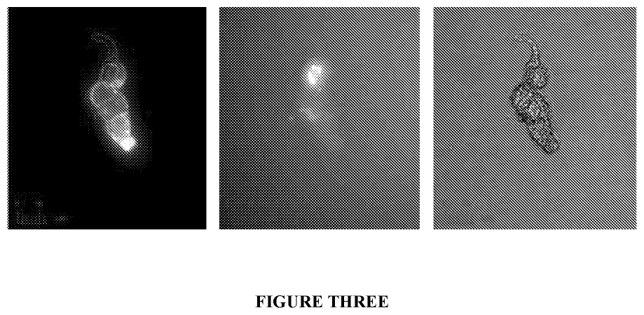

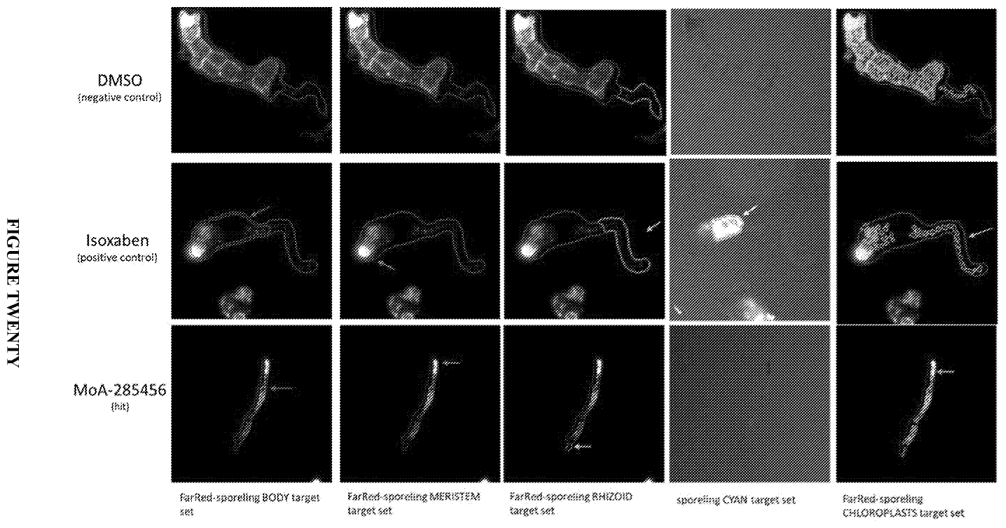

Images were processed individually using the software Developer. Image analysis protocols were designed to distinguish plants and subcellular objects such as nuclei from the background (FIGS. 3 and 20).

When multiple images were produced the segmented objects could be linked so that any object segmented using one image was parent to a multitude of objects segmented using another image. 10-50 measurements that described the morphological and physiological phenotype of the plants were then extracted. A non-exhaustive list of measurements was, for example, “plant length”, “plant width”, “plant circularity”, “chlorophyll fluorescence intensity”, and “number of cells per plant”. Measurements were finally recorded in .csv or .txt files for each plant in each well imaged.

Data Formatting

The output files were reformatted for downstream analysis compatibility using custom parsing scripts. The scripts automatically added relevant metadata such as the barcode of assay plates analysed and/or the dates and protocols of image acquisition and image analysis. Furthermore, the script for high resolution analysis output files (4X and 10X datasets) introduced a default value+/−an error factor for variables that took a value only if a Cyan object, a Far-Red object or both a Cyan and a Far-Red object were defined when the parent Brightfield object was not linked to a Cyan, Far-Red or both a Cyan and Far-Red object respectively.

Data Preparation

Data analysis was carried out using the HC StratoMineR software developed by CoreLifeAnalylitcs. The software provided a data analysis pipeline where each step could be customized by changing the statistical methods and parameters used. The steps, methods and parameters used in this Example are provided below:

Data was normalized by the median of the negative (DMSO) controls at the plate level to minimise noise across plates. The distribution of variables was checked for normality and transformed, if necessary, following the automated recommendation of the software. Data transformation methods could also be manually chosen from the following list: square root, power of 2, power of 3, log, log 2, log 10, inverse. Variables were then scaled to equalize the weight of variables with different means in downstream analysis steps. Scaling was carried out using the Z-score method at the plate level (FIG. 5). Screen level scaling was also possible at this step when the number of data points per plates was too low for the downstream analysis steps to run accurately.

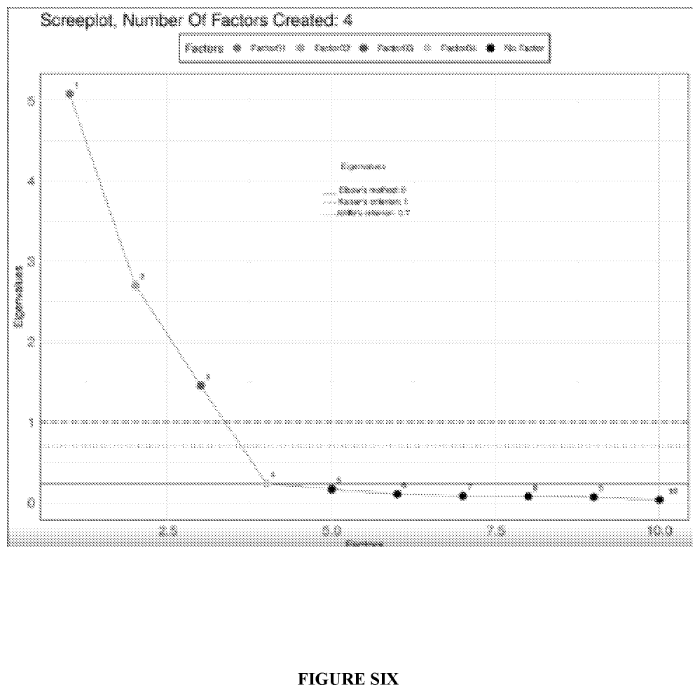

Correlated variables in the negative control and screen compound data were reduced to factors by Common Factor Analysis set to run with 200 t-SNE iterations, t-SNE perplexity set as 30, oblique rotation method and factor scoring method as ten Berge. The number of factors retained may be automatically determined according to the Kaiser's Elbow or Joliffe's criterion, or manually selected upon visual inspection of a scree plot (FIG. 6).

Hit Selection

Unsupervised Hit Selection

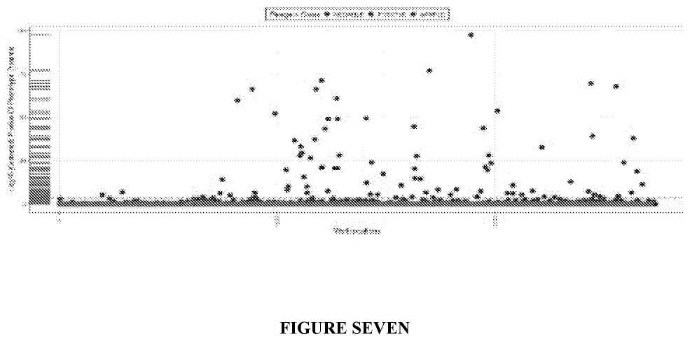

Screen compounds were selected as hits according to the phenotypic Euclidean distance to the median of the negative control in the multifactorial space defined by the factors previously retained. A p-value of 0.0001 was chosen as a significance threshold above which screen compounds were considered significantly different from the median of the negative controls (FIG. 7).

Artificial Intelligence Supervised Hit Selection

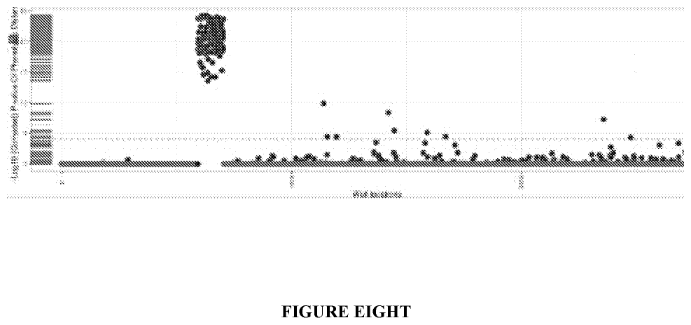

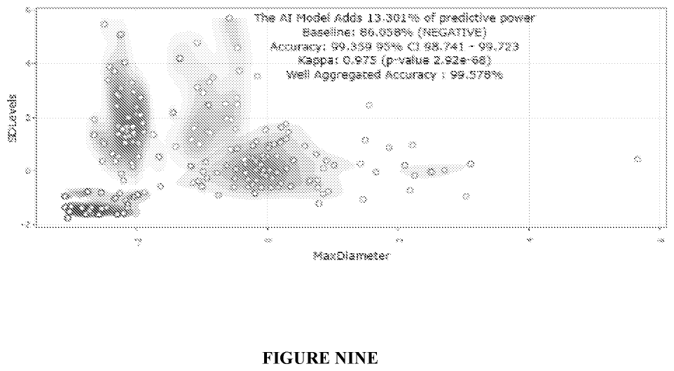

Screen compounds were selected as hits according to their dissimilarity to the phenotypic model of the negative control (FIG. 8). The phenotypic models for the negative controls and positive controls were generated by a Random Forest algorithm with 128 trees using 80% of the corresponding dataset for training and the remaining 20% for testing the generated models (FIG. 9).

Known or Unknown Mode of Action Prediction

Unsupervised Mode of Action Prediction

The mode of action of hits selected with the unsupervised method was predicted by the clustering of hits with positive controls. Positive controls were commercial herbicides used at concentrations known to cause specific mode of action symptoms. The concentration of commercial herbicides known to cause specific mode of action symptoms was determined experimentally by visual inspection of dose-response experiments where plants were exposed to a range of herbicide concentrations.

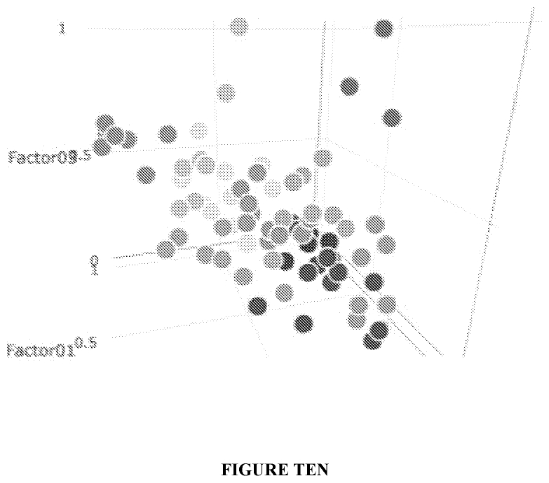

Clusters of hits and positive control standards were generated using the Ward agglomerative method where K-means are automatically selected and the distance between cluster centers of gravity was calculated as the Euclidean distance in the multifactorial space applying a significance threshold of p-value=0.0001 (FIG. 10).

On average, the automated classification of hits into clusters was 86% consistent with manual classification of hits into groups of phenotypic similarity. If the hit fell outside of clusters associated with a known mode of action positive control, the hit was visually inspected for the presence of unique symptoms and if unique symptoms were observed the hit was predicted to have a novel mode of action.

If the hit could not be predicted to be either a known or novel mode of action, the hit was manually progressed to a dose-response experiment where plants were treated with a range of concentrations of the hit ranging from 1 nM to 50000 nM and all resulting datapoints were processed according to the method previously described from Image analysis onwards.

AI Supervised Mode of Action Prediction

A statistical test was carried out to determine the probability that a hit matched a phenotypic model (any of the positive controls). If the probability was higher than an arbitrarily defined threshold that depended on the number of positive control phenotypic models, the hit was predicted to have a known mode of action and this mode of action to be the mode of action of the herbicide which phenotypic model the hit is most strongly associated to. This decision-making step was currently carried out manually but could be automated by a custom parsing script.

If the hit fell below the arbitrarily defined significance threshold for all positive control phenotypic models, the hit was visually inspected for the presence of unique symptoms and if unique symptoms were observed the hit was predicted to have a novel mode of action.

If the hit could not be predicted to have either a known or novel mode of action, the hit was progressed to a dose-response experiment where plants were treated with a range of concentrations of the hit ranging from 1 to 50000 nM and all resulting datapoints are processed according to the method described from Image analysis onwards.

### Discussion

High content screening has not been applied to herbicide discovery screens to date. The state of the art relative to high content analysis applied to herbicide discovery and simultaneous mode of action prediction has been limited to a few modes of action (an automated quantitative image analysis tool for the identification of microtubule patterns in plants). Furthermore, these methods require higher resolution (confocal microscopy) and as a consequence rely on explants or subparts of plants rather than a miniature or whole plant screening system.

By contrast, the present method is applicable to more modes of action and uses miniature plants because it does not rely on confocal microscopy. As a consequence, the method of the present invention is higher throughput and has a broader scope.

Due to the lack availability of screening systems using miniature or whole plants, it was non-obvious that a high content screening approach could be applied to a herbicide discovery campaign.

### Example Three: Identification of Mutants Resistant to Herbicide Hits

### Background

Knowledge of targets informs mode of action, toxicity, resistance breaking and further screening or lead optimisation efforts. Mutations in genes encoding protein targets may confer resistance to herbicide. Reverse identification of target genes was therefore trialed by the inventors of the present invention.

Mutagenised microspores were a convenient system to screen for herbicide resistance conferring mutations because of their small size and amenability to simple irradiation mutagenesis methods. Herbicide resistance screening following spore mutagenesis has not been reported previously in Marchantia.

Methods

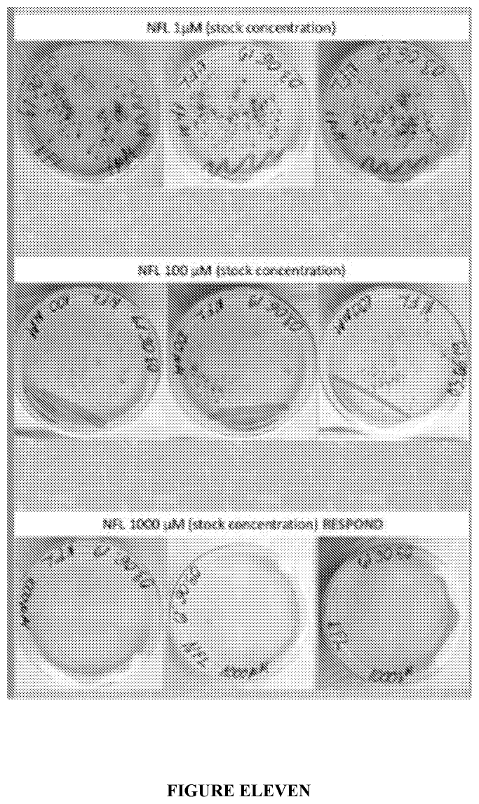

A lethal concentration of the herbicide hit was determined in the condition of the mutagenesis concentration. 20000 Marchantia spores were plated in 90 mm Petri dishes containing 25 mL of Johnson's medium containing 1.4% agar and supplemented with a range of herbicide hit concentrations ranging from 1 to 50000 nM. The lethal concentration was defined as the minimum concentration of herbicide hit sufficient to kill 100% of the wild type Marchantia plants (FIG. 11).

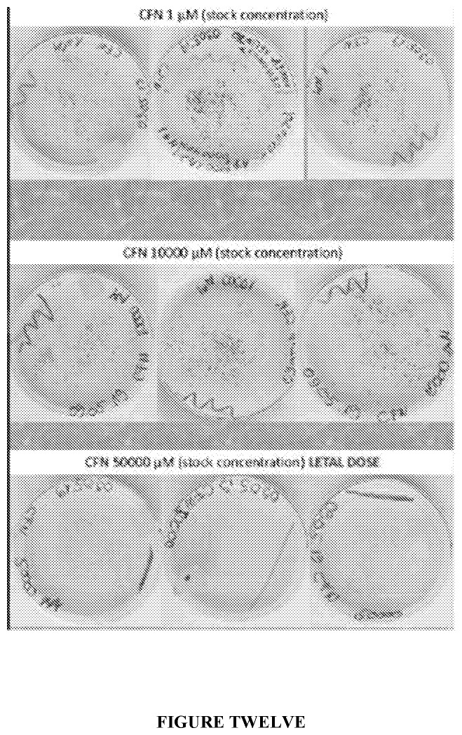

If no lethal concentration was observed, the highest concentration would be used in place of the lethal concentration provided that it caused the plants to show a phenotype distinct from the phenotype of untreated plants. For example, this alternative phenotype may be a strong reduction in growth (FIG. 12) or a marked change in the shape of the plants, or a change in plant pigmentation.

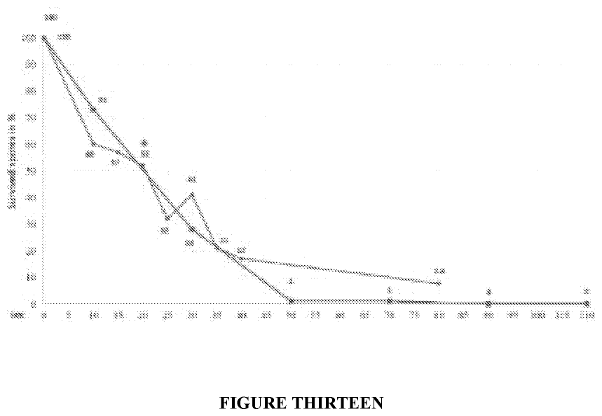

Mutagenised populations of Marchantia spores were generated using either physical or chemical mutagens at a dose that kills an arbitrarily defined proportion of the mutagenised population of spores (FIG. 13). The arbitrarily defined proportion of the mutagenised population of spores that defined the experimental mutagen dose could be 50% as is standard in the field, or higher or lower depending on the phenotype of wild type plants treated with the herbicide hit at the mutagenesis screen concentration.

A population of 400000 or higher mutagenised spores was spread out between 20 90 mm Petri dishes containing 25 mL of Johnson's medium containing 1.4% agar and supplemented with the herbicide hit at a concentration 10-fold higher than the lethal concentration. Alternatively, 400000 or higher wild type spores were spread out between 20 90 mm Petri dishes containing 25 mL of Johnson's medium containing 1.4% agar and supplemented with the herbicide hit at a concentration 10-fold higher than the lethal concentration, and the wild type spores were then mutagenised.

One example of the mutagenesis method is UV-B mutagenesis. 400000 spores were spread out on 20 90 mm Petri dishes containing 25 mL of Johnson's medium containing 1.4% agar and supplemented with a 10-fold higher concentration of herbicide than the lethal concentration. Petri dishes were then inserted upside down in a UV-B transilluminator with the lid removed with the spores directly facing directly the UV-B light source. UV-B light was shone upon the spores for the duration required to reach the desired mutagenesis dose. Petri dishes were then closed and wrapped in aluminum foil to exclude light and the Petri dishes placed in a 23 Celsius degrees incubator overnight.

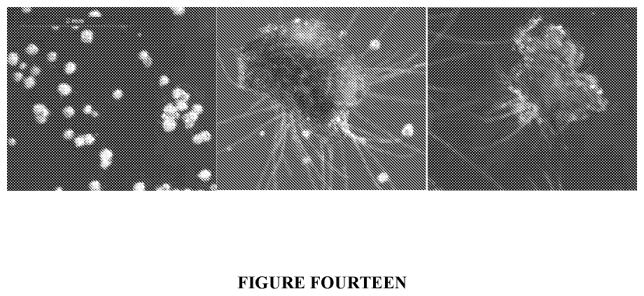

Mutagenised spores in Petri dishes containing the herbicide hit at the desired concentration were placed in an incubator with constant illumination, lux and 23° C. and plants were grown for 14 days. Survivors (FIG. 14), or otherwise untreated-looking plants, were transferred to a new petri dish in the absence of the herbicide hit to grow for another 14 days. Herbicide resistance was validated by transferring a fragment of the grown plants to a fresh Johnson's media containing 1.4% agar supplemented with a concentration 10-fold higher than the lethal concentration.

If the resistance phenotype was validated, genomic DNA was extracted from any part of or the whole mutant plant using any DNA extraction method such as but not limited to Phenol-Chloroform-IAA extraction. A genomic DNA library was prepared and sequenced using any Illumina Next-Generation Sequencing more recent than Hi Seq 2000.

### Example Four: Identification of Causative Mutation in the RHO GTPASES of PLANTS ENHANCER PROTEIN Gene Impairing Fertility (Case B)

In this Example, the methodology of the invention is used to identify a causative mutation associated with rhizoid/epidermis phenotype in Marchantia polymorpha, as detailed below. These methods are equally applicable to determining phenotypes associated with herbicide resistance or plant growth regulation.

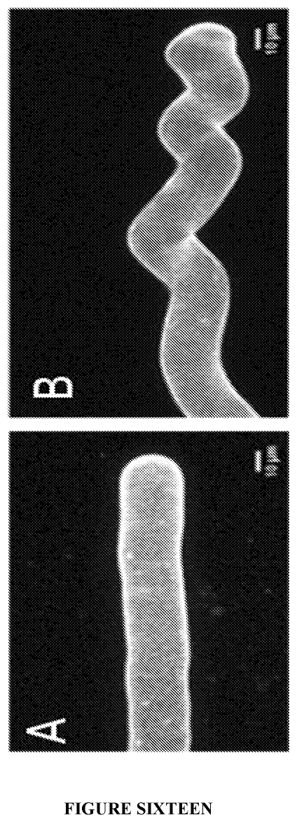

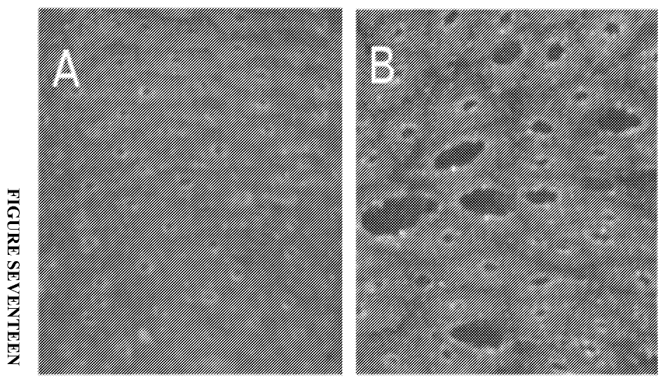

Several independent mutant lines were generated by irradiating Marchantia polymorpha spores with ultraviolet B. Mutants lines were classified into two phenotypic groups: some had straight rhizoids (FIG. 16A) and intact epidermis (FIG. 17A), some had wavy rhizoids (FIG. 16B) and stretched epidermis (FIG. 17B).

We aimed to identify the causative mutation in the UV4.32 mutant line, which has wavy rhizoids and stretched epidermis. DNA was extracted from a UV4.32 mutant with wavy rhizoids and stretched epidermis using the whole plant as a sample and standard DNA PhenolChlorophorm-IAA extraction. The genomes of UV4.32 and the genome of 7 independent mutant lines with straight rhizoids and intact epidermis were sequenced using Illumina's HiSeq-2000 platform technology.

Raw reads were quality trimmed using Trimmomatic-0.32 and normalised using Khmer0.7.1 with a k-mer size of 31. Resulting reads were aligned against the reference genome using bowtie2-2.1.0 set in —very-sensitive-local mode. The reference genome used is a draft Marchantia polymorpha genome assembly publicly available on the NCBI Whole Genome Shotgun (WGS) database.

Alignments were position sorted and mismatches within reads with q quality higher than 35 were extracted using the function sort and mpileup from bio-samtools-2.0.5. Because they were likely caused by misalignments, mismatches in regions with coverage exceeding 100× were excluded using the varFilter function from bcftools of the samtools-0.1.9 package. Then, mismatches were retained only if they were supported by more than 7 reads and if they appeared sufficiently homozygous based on a negative FQ value or AF1 value higher than 0.5001.

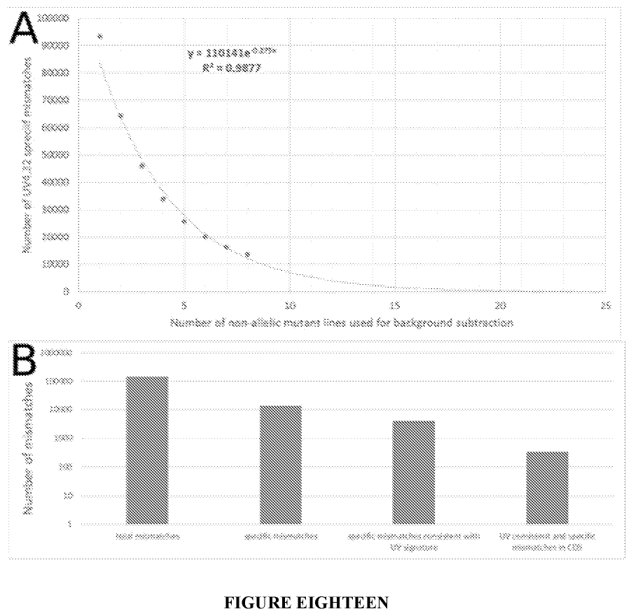

In total, 143 292 mismatches were identified in UV4.32 before any filtering. The number of mismatches specific to UV4.32 decreased with the number of UV mutant lines with straight rhizoids and intact epidermis used for filtering (FIG. 18A).

Ultimately, using all filtering lines sequenced, the number of candidate mismatches was reduced to 12 000 mismatches, or more than 90% decrease (FIG. 18B). This shows that filtering step of subtracting the set of mismatches in the test sample by the set of mismatches in comparison samples that are predicted not to harbour the causative mutation increased the stringency of candidate mismatches identification, prior to standard filtering steps.

Subsequent filtering steps were performed to filter for mismatches inconsistent with UV signature, filter for mismatches outside the gene coding sequence and to filter for nonsynonymous mismatches. These three filtering steps further reduced the number of candidate mismatches to 10 mutations that were consistent with the expected UV mutation signature (FIG. 18), were predicted to be in the coding sequence of a gene (FIG. 18) and to change the amino acid sequence of the corresponding protein (Table 1).

TABLE 1
Candidate mutations for UV4.32 in Marchantia genes and corresponding
Arabidopsis homologous genes. Arabidopsis thaliana is the
most established model for plant genetics and the function
of Marchantia polymorpha genes may be inferred by analogy
with the function of Arabidopsis genes.

| | | Annotation of | |
|---|---|---|---|
| Mutated gene | Arabidopsis | Arabidopsis | Type of |
| model | homolog | homolog | mutation |
| MpREN | AT5G12150/ | Rho GTPase | Frame shift, |
| | PHGAP1 | Activating Protein | early stop |
| Mp1660s1160 | AT5G38840 | SMAD/FHA domain | missense |
| | | containing protein | |
| Mp2415s1240 | AT2G29510 | hypothetical protein | missense |
| | | DUF3527 | |
| Mp2490s1660 | AT1G74410 | RING protein | missense |
| Mp2782s1160 | None | NA | missense |
| Mp3036s1070 | AT3G54750 | unannotated | missense |
| Mp3802s1070 | AT1G06560 | NOP2C, RNA | missense |
| | | methylation | |
| Mp4605s1070 | None | NA | missense |
| Mp773s1730 | AT1G73060 | LPA3 | missense |
| Mp909s1190 | AT2G07360 | SH3 domain- | missense |
| | | containing protein | |

Of the 10 mutations, the strongest mutation is a 2 base pair deletion causing an early stop codon in MpREN (Table 1). Ren mutants are known to exhibit the same phenotype as UV4.32 (Honkanen et al, 2016). This suggests that the subsequent filtering steps were sufficiently conservative.

Altogether, this shows that the version of our pipeline based on subtracting the set of mismatches in the test sample by the set of mismatches in comparison samples that are predicted not to harbour the causative mutation enables the identification of a small number of mutations, including the causative mutation, without needing to outcross the mutant lines.

### Example Five: Discovery of Mutations in the ACETOLACTATE SYNTHASE Gene Causing Chlorsulfuron Resistance (Case A)

Marchantia polymorpha spores were irradiated with ultraviolet B irradiation and seven independent mutant lines resistant to the herbicide chlorsulfuron were identified. Chlorsulfuron resistance was determined by a Marchantia polymorpha plant that was alive two weeks following exposure to a lethal dose of Chlorsulfuron (0.1 ppm dose, i.e. a dose sufficient to kill 100% of wild-type plants).

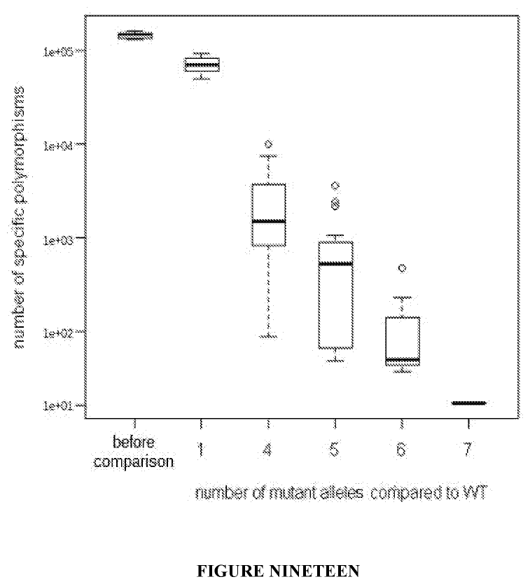

Since all mutant plants shared the same phenotype—chlorsulfuron resistance—we hypothesised that they each harbour the same causative mutation. Comparing the chlorsulfuron resistant mutants to the reference genome individually identified over 100 000 mismatches and we first filtered our mismatches that were also present in a M0 wild type genome (FIG. 19, the 2 left-most scatter boxes).

To test the efficiency of the allelism-based version of our pipeline, we applied it to combinations of 4, 5, 6, and all 7 chlorsulfuron mutants. The more allelic subtracting lines we use, the more efficient the pipeline becomes. In fact, using all 7 chlorsulfuron resistant lines, we decreased the number of mismatches from nearly 100 000 to 11 candidate mutations that are consistent with the expected mutational signature and are in the coding sequence of a gene (FIG. 19).

Of the 11 candidate mutations that are common to all 7 chlorsulfuron resistant mutants but absent from wild type, 5 cause a change in the amino acid sequence of the coded protein (Table 2). Of those 5 candidate mutations, only one is in a gene with a predicted function. In fact, this exact mutation in the acetolactate synthase gene is known to cause chlorsulfuron resistance in other plant models.

TABLE 2
Candidate mutations for chlorsulfuron mutants (case A)

| | | Annotation of | |
|---|---|---|---|
| Mutated gene | Arabidopsis | Arabidopsis | Type of |
| model | homolog | homolog | mutation |
| Mp3229s1050 | None | NA | nonsense |
| Mp2743s1010 | None | NA | nonsense |
| Mp3364s1000 | None | NA | missense |
| Mp4485s1300 | None | NA | missense |
| Mp2116s1050 | AT3G42690 | Acetolactate | missense |
| | | synthase | |

### Example Six: Discovery of Mutations in the ACETOLACTATE SYNTHASE Gene Causing Chlorsulfuron Resistance (Case AB)

To improve the power of the pipelines exemplified in Example 1 and Example 2, we combined both approaches: in this embodiment of the pipeline, the causative mutations is looked for in the group of mismatches that are common to allelic mutants and absent from wild type and non-allelic mutants.

Using 3 chlorsulfuron sensitive mutagenized lines, we filtered 4 of the 11 chlorsulfuron resistant specific mismatches previously identified as being consistent with the expected mutational signature and being in the coding sequence of a gene, finally leaving us with only 4 candidate mutations (Table 3) predicted to cause a change in the amino acid sequence of a protein.

This represents a 20-30% increase in the power of the pipeline compared the pipeline exemplified in Example 2 alone. Because the power of the pipeline in Example 1 and 2 increases with the number of allelic and non-allelic subtracting lines resp., we predict that the power of the pipeline exemplified in the present Example will increase further if we use more allelic and non-allelic subtracting lines.

TABLE 3
Candidate mutations predicted to cause a change
in the amino acid sequence of a protein.

| | | Annotation of | |
|---|---|---|---|
| Mutated gene | Arabidopsis | Arabidopsis | Type of |
| model | homolog | homolog | mutation |
| Mp2743s1010 | None | NA | nonsense |
| Mp3364s1000 | None | NA | missense |
| Mp4485s1300 | None | NA | missense |
| Mp2116s1050 | AT3G42690 | Acetolactate | missense |
| | | synthase | |

1. A method of screening candidate compounds for herbicidal activity or plant growth regulating activity, the method comprising the steps of:
(i) contacting a series of different candidate compounds with a plurality of test samples from non-vascular plants; and
(ii) determining whether the test samples provide a phenotypic response to said series of different candidate compounds by comparison to phenotypes of control samples from non-vascular plants not contacted with candidate compounds;
wherein the test samples and the control samples comprise whole-plants, spores, sporelings, explants, protoplasts or vegetative propagules, and the phenotypic response is indicative of the herbicidal activity or the plant growth regulating activity.

2. The method according to claim 1, wherein the candidate compounds are candidate compounds for herbicidal activity.

3. The method according to claim 1, wherein the non-vascular plant is a moss, homwort or liverwort.

4. The method according to claim 1, wherein the test samples and control samples are sporelings.

5. The method according to claim 4, wherein the test and control sporelings originate from spores of the same species of non-vascular plant.

6. The method according to claim 4, wherein the test sporelings are moss sporelings, liverwort sporelings, homwort sporelings, or any combination thereof.

7. The method according to claim 1, wherein each member of the series of different candidate compounds is contacted with a different test sample.

8. The method according to claim 1, wherein multiple members of the series of different candidate compounds are contacted with a single test sample.

9. The method according to claim 1, wherein the test samples and control samples are leafy liverwort sporelings, simple thalloid liverwort sporelings, complex thalloid liverwort sporelings, or any combination thereof.

10. The method according to claim 1, wherein the test samples and control samples are selected from the group consisting of: Marchantia alpestris sporelings, Marchantia aquatica sporelings, Marchantia berteroana sporelings, Marchantia carrii sporelings, Marchantia chenopoda sporelings, Marchantia debilis sporelings, Marchantia domingenis sporelings, Marchantia emarginata sporelings, Marchantia foliacia sporelings, Marchantia grossibarba sporelings, Marchantia inflexa sporelings, Marchantia linearis sporelings, Marchantia macropora sporelings, Marchantia novoguineensis sporelings, Marchantia paleacea sporelings, Marchantia palmata sporelings, Marchantia papillate sporelings, Marchantia pappeana sporelings, Marchantia polymorpha sporelings, Marchantia rubribarba sporelings, Marchantia solomonensis sporelings, Marchantia streimannii sporelings, Marchantia subgeminata sporelings, Marchantia vitiensis sporelings, Marchantia wallisii, Marchantia nepalensis, and any combination thereof.

11.-16. (canceled)

17. The method according to claim 1, wherein step (ii) comprises comparing phenotypes of the test samples to phenotypes of positive control samples contacted with known herbicidal or plant growth regulator compounds and further comprises comparing phenotypes of the test samples to phenotypes of negative control samples not contacted with known herbicidal or plant growth regulator compounds.

18. The method according to claim 17, wherein the known herbicidal compounds have a known mode of action, and said comparing of test sample phenotypes to positive control sample phenotypes is used to predict the mode of action of a candidate compound identified to have herbicidal or plant growth regulating activity.

19.-21. (canceled)

22. The method according to claim 1, wherein step (ii) comprises obtaining measurements of any one or more of: sample length, sample width, sample shape, sample pigmentation, sample circularity, sample chlorophyll concentration, and/or number of cells per sample.

23.-27. (canceled)

28. The method according to claim 1, wherein the method further comprises step (iii) of:
(a) contacting a candidate compound identified to have herbicidal or plant growth regulating activity in steps (i) and (ii) with a series of mutagenized samples comprising whole-plants, spores, sporelings, explants, protoplasts or vegetative propagules, wherein the test and mutagenised samples are from the same species of non-vascular plant;
(b) extracting DNA from a resistant mutagenised sample which survives said contacting in (a), or which does not exhibit growth abnormalities after said contacting in (a);
(c) sequencing the genome or a genomic portion of the resistant mutagenised sample to thereby obtain a mutagenised sample DNA sequence;
(d) aligning the mutagenized DNA sequence obtained in (c) to a reference DNA sequence and identifying a first set of sequence mismatches between the mutagenised sample DNA sequence and the reference DNA sequence;
(e) aligning a DNA sequence from a first comparison sample to said reference DNA sequence and identifying a second set of mismatches between the first comparison DNA sequence and the reference DNA sequence; and
(f) filtering the first set of mismatches with respect to the second set of mismatches to identify a first subset of mismatches that are unique to the first set of mismatches, wherein the first subset of mismatches are candidate mutations that may confer resistance to herbicides or to plant growth regulators;
wherein the first comparison sample is from an independent sample that does not survive contacting with the candidate compound or which exhibits growth abnormalities after contacting the candidate compound, and is of the same genus as the resistant mutagenised sample, and wherein the reference DNA sequence is a known reference sequence of a plant of said genus.

29. The method according to claim 28, wherein the method further comprises:
(e-i) aligning a DNA sequence of a second comparison sample to the reference DNA sequence and identifying a third set of mismatches between the second comparison sample and reference DNA sequence; and
(f) filtering the first set of mismatches with respect to the third set of mismatches to facilitate identification of a second subset of mismatches that are unique to the first set of mismatches, and generating a third subset of mismatches by filtering the first subset of mismatches with respect to the second subset of mismatches, wherein the first and second subsets of mismatches are candidate mutations that may confer resistance to herbicides or resistance to plant growth regulators;
wherein the second comparison sample is from an independent sample that does not survive contacting with the candidate compound or which exhibits growth abnormalities after contacting the candidate compound, and is of the same genus as the mutagenised samples.

30. The method according to claim 28, wherein the mutagenised samples are M1 samples.

31. (canceled)

32. The method according to claim 28, wherein the method does not comprise a step of segregation analysis, complex segregation analysis or bulk segregation analysis.

33.-34. (canceled)

35. The method according to claim 28, wherein the mutagenised samples are haploid.

36. The method according to claim 28, wherein the candidate mutations are in a gene encoding a protein that is targeted by the candidate compound identified to have herbicidal or plant growth regulating activity.

37. The method according to claim 28, wherein step (iii) is implemented using a computer.

38. (canceled)
